SEQUENCE LISTING (3 sequences)

SEQ ID NO: 1 - protein, 904 aa, Hordeum vulgare subsp. vulgare
MAVGETGASVSAAEAEAEATQAFMPDARAYWVTSDLIAWNVGELEAQSVCLYASRAAAMSLSPSNGGIQGYDSKVELQPESAGLPETVTQKFPFISSYRAFKVPSSVDVASLVKCQLVVASFGADGKHVDVTGLQLPGVLDDMFAYTGPLGAVFSEDSVSLHLWAPTAQGVSVCFFDGPAGPALETVQLKESNGVWSVTGPREWENRYYLYEVDVYHPTKAQVLKCLAGDPYARSLSANGARTWLVDINNETLKPASWDELADEKPKLDSFSDITIYELHIRDFSAHDGTVDSDSRGGFRAFAYQASAGMEHLRKLSDAGLTHVHLLPSFHFAGVDDIKSNWKFVDECELATFPPGSDMQQAAVVAIQEEDPYNWGYNPVLWGVPKGSYASDPDGPSRIIEYRQMVQALNRIGLRVVMDVVYNHLDSSGPCGISSVLDKIVPGYYVRRDTNGQIENSAAMNNTASEHFMVDRLIVDDLLNWAVNYKVDGFRFDLMGHIMKRTMVTKSALQSLTTDAHGVDGSKIYLYGEGWDFAEVARNQRGINGSQLNMSGTGIGSFNDRIRDAINGGNPFGNPLQQGFNTGLFLEPNGFYQGNEADTRRSLATYADQIQIGLAGNLRDYVLISHTGEAKKGSEIHTFDGLPVGYTASPIETINYVSAHDNETLFDVISVKTPMILSVDERCRINHLASSMMALSQGIPFFHAGDEILRSKSIDRDSYNSGDWFNKLDFTYETNNWGVGLPPSEKNEDNWPLMKPRLENPSFKPAKGHILAALDSFVDILKIRYSSPLFRLSTANDIKQRVRFHNTGPSLVPGVIVMGIEDARGESPEMAQLDTNFSYVVTVFNVCPHEVSMDIPALASMGFELHPVQVNSSDTLVRKSAYEAATGRFTVPGRTVSVFVEPRC

SEQ ID NO: 2 - protein, 555 aa, Geobacillus stearothermophilus
MKKTWWKEGVAYQIYPRSFMDANGDGIGDLRGIIEKLDYLVELGVDIVWICPIYRSPNADNGYDISDYYAIMDEFGTMDDFDELLAQAHRRGLKIILDLVINHTSDEHPWFIESRSSRDNPKRDWYIWRDGKDGREPNNWESIFGGSAWQYDERTGQYYLHLFDVKQPDLNWENSEVRQALYDMINWWLDKGIDGFRIDAISHIKKKPGLPDLPNPKGLKYVPSFAAHMNQPGIMEYLRELKEQTFARYDIMTVGEANGVTVDEAEQWVGEENGVFHMIFQFEHLGLWKRKADGSIDVRRLKRTLTKWQKGLENRGWNALFLENHDLPRSVSTWGNDREYWAESAKALGALYFFMQGTPFIYQGQEIGMTNVQFSDIRDYRDVAALRLYELERANGRTHEEVMKIIWKTGRDNSRTPMQWSDAPNAGFTTGTPWIKVNENYRTINVEAERRDPNSVWSFYRQMIQLRKANELFVYGAYDLLLENHPSIYAYTRTLGRDRALIIVNVSDRPSLYRYDGFRLQSSDLALSNYPVRPHKNATRFKLKPYEARVYIWKE

SEQ ID NO: 3 - protein, 459 aa, Agrobacterium sp. H13-3
MTDHKALAARFPGDFLFGVATASFQIEGATKVDGRKPSIWDAFCNMPGHVFGRHNGDVACDHYNRWEDDLDLIKEMGVEAYRFSIAWPRIIPDGFGPINEKGLDFYDRLVDGCKARGIKTYATLYHWDLPLTLMGDGGWASRSTAHAFQRYAKTVMARLGDRLDAVATFNEPWCAVWLSHLYGIHAPGERNMEAALAAMHHINLAHGFGVEASRHVAPKVPVGLVLNAHSVIPASNSDADMKAAERAFQFHNGAFFDPVFKGEYPAEMMEALGSRMPVVEAEDLSIISQKLDWWGLNYYTPMRVADDATEGAEFPATKQAPAVSDVKTDIGWEVYAPALHSLVETLYERYELPDCYITENGACYNMGVENGEVDDQPRLDYYAEHLGIVADLVKDGYPMRGYFAWSLMDNFEWAEGYRMRFGLVHVDYETQVRTLKNSGKWYSALASGFPKGNHGVMKG

(19) United States
(12) Patent Application Publication
(10) Pub. No.: US 2015/0140573 A1
(43) Pub. Date: May 21, 2015

(54) ASSAYS FOR DETECTING GLUCOSIDASE ACTIVITY

(71) Applicant: Megazme International Ireland, Bray, County Wicklow (IE)

(72) Inventors: Marie Bøjstrup, Taastrup (DK); Caspar Elo Christensen, Vanlose (DK); Ole Hindsgaul, Copenhagen V (DK)

(21) Appl. No.: 14/401,756

(22) PCT Filed: Jun. 7, 2013
(86) PCT No.: PCT/DK2013/050183

§ 371 Date: Nov. 17, 2014

(30) Foreign Application Priority Data
Jun. 12, 2012 (DK) PA 2012 70321

(51) Int. Cl. C12Q 1/34; C12Q 1/40; G01N 33/58

(52) U.S. Cl. CPC C12Q 1/34; G01N 33/582; C12Q 1/40; G01N 2333/944
USPC 435/7.4; 435/18; 536/17.8; 435/22

(57) ABSTRACT

The present invention relates to a method of detecting α-(1→6)-glucosidase activity in a sample. The method is for example useful for determining the limit dextrinase activity in a sample. The method involves use of an oligosaccharide substrate of the formula X-(glucoside)n-*(glucoside)m-Z—Y, where X is a blocking group, -* is a α-(1→6)-glucosidic linkage and Y is a detectable label. Upon cleavage of the α-(1→6)-glucosidic linkage, the detectable label is released and thus the α-(1→6)-glucosidase activity can be determined. The invention also relates to the oligosaccharide substrate per se.

### FIELD OF INVENTION

The present invention relates to the field of detection of glucosidase activity and more particular to detection of α-(1→6)-glucosidase activity. In a preferred embodiment the invention relates to detection of limit dextrinase activity. The Limit Dextrinase activity may be detected in any composition, for example in extracts of plants or in plant products.

### BACKGROUND OF INVENTION

Limit dextrinase (LD) is a starch debranching enzyme, cleaving the α-(1→6)-glycosidic bonds in branched dextrins. The presence of this enzyme has been shown to be of major importance in order to achieve a high degree of fermentation in the brewing process (McGregor, A. W; Bazin, S. L; Macri, L. J; Babb, J. C. J. Cereal Sci. 1999, 29, 161; Stenholm, K; Home, S. J. I. Brewing, 1999, 105, 205). It is therefore highly desirable for brewers to have an easy and sensitive method to determine the amount of active limit dextrinase in malt and during processing. The assay should be specific to limit dextrinase, as a range of other glycoside hydrolases, such as α-amylase, β-amylase and α-glucosidases, are also present in malt. Only few methods for detection of limit dextrinase have been developed. The most widely used assay for limit dextrinase detection is the Limit Dextrizyme kit from Megazyme based on a dye-labelled cross linked pullulan (McCleary, B. V. Carbohyd. Res, 1992, 227, 257.). With this method centrifugation has to be performed before measuring the degree of hydrolysis, making it impossible to follow the hydrolysis in real time. Substantial sample amount has to be used involving tedious centrifugations. Furthermore the method is rather insensitive and therefore dependent of added reducing agents to increase the activity of limit dextrinase in the malt extract.

### SUMMARY OF INVENTION

Accordingly, there is an unmet need for specific assays to specifically determine α-(1→6)-glucosidase activity and in particular limit dextrinase activity in a sample in real time, where the sample may also comprise other glycoside hydrolases.

The present invention provide an oligosaccharide substrate useful for determining α-(1→6)-glucosidase activity and in particular limit dextrinase activity in a sample in real time, where the sample may also comprise other glycoside hydrolases. In particular the invention provides an oligosaccharide substrate of the formula I

X-(glucoside)ₙ-*(glucoside)ₘ-Z—Y
wherein
X is a blocking group capable of inhibiting cleavage by an exo-glucosidase; and
Y is a detectable label; and
Z is either S or O or N; and
-* is a α-(1→6)-D-glucosidic linkage; and
n and m individually are integers in the range of 1 to 6;

The invention also provides methods of detecting α-(1→6)-glucosidase activity in a sample, the method comprising the steps of
a. Providing a sample
b. Providing an oligosaccharide substrate of the formula

X-(glucoside)ₙ-*(glucoside)ₘ-Z—Y
wherein
X is a blocking group capable of inhibiting cleavage by an exo-glucosidase; and
Y is a detectable label; and
Z is either S or O or N; and
-* is a α-(1→6)-glucosidic linkage; and
n and m individually are integers in the range of 1 to 6;
c. Optionally, providing at least one exo-glucosidase
d. Incubating the sample with the oligosaccharide substrate and optionally with the exo-glucosidase
e. Determining the presence of free detectable label
   wherein the presence of free detectable label is indicative of α-(1→6)-glucosidase activity in said sample.

### DESCRIPTION OF DRAWINGS

FIG. 1 shows a scheme for preparing an example of an oligosaccharide substrate according to the invention, namely compound 6. a) denotes acetic anhydride, pyridine, DMAP, rt, ON; b) denotes HBr (33% in acetic acid), DCM, rt; c) denotes 4-hydroxy-3-nitrophenylacetic acid methyl ester, TBABr, DCM, NaHCO₃ (1M), KCl (1M), 45° C., ON; d) denotes NaOMe (1M), MeOH, rt, ON; e) denotes Benzaldehyde dimethyl acetal, CSA, DMF, 60° C. on the right hand side the chemical structure of the compound designated compound 6 is shown.

FIG. 2 shows the hydrolysis of compound 6 in barley malt extract and extract spiked with limit dextrinase.

FIG. 3 shows the hydrolysis of compound 6 in barley malt extract and extract inhibited with limit dextrinase inhibitor.

FIG. 4 shows the hydrolysis of compound 6 mediated by α-amylase from A. oryzae relative to limit dextrinase (LD).

FIG. 5 shows a scheme for the synthesis of an example of a TAMRA labeled oligosaccharide compound 9 for enzymatic studies. a) Propargylamine, RT, 3 days, then Ac₂O, MeOH, over night; b) TAMRA-azide 8, CuSO₄, sodium ascorbate, 50° C., 10 min.

FIG. 6 shows MALDI-ToF mass-spectra (HCCA as matrix) of products remaining after hydrolysis of the TAMRA-heptasaccharide (compound 9) (m/z 1744.9) to maltotriose-TAMRA (m/z 1096.7) mediated by various hydrolytic enzymes.

FIG. 7 shows a scheme for synthesis of FRET substrate 14. a) CSA, DMF, 4 Å MS; b) CuSO₄, sodium ascorbate, TAMRA-azide 8, 50° C., 10 min; c) DBU in DMF then BHQ-2 NHS (compound 13) in phosphate buffer pH 7.6.

FIG. 8 shows the time-dependent increase in fluorescence when the bifunctionally labeled substrate 14 is hydrolyzed with recombinant barley Limit Dextrinase.

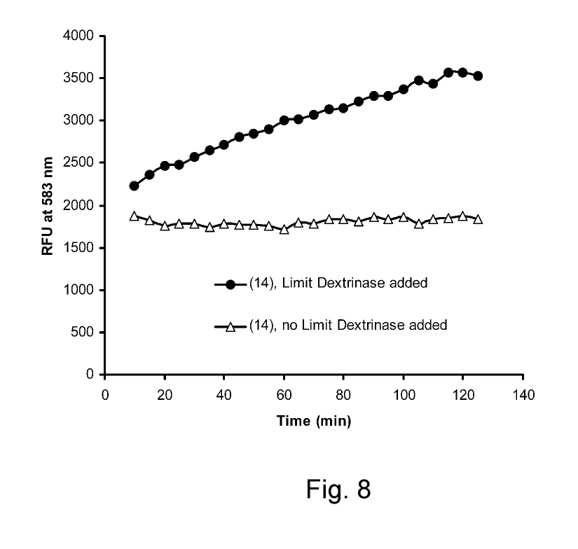

FIG. 9 shows a scheme for the synthesis of compound 10. a) K₂CO₃, DMF, 80° C.; b) H₂, Pd/C (10% Pd), MeOH, room temperature; c) FmocCl, dioxane, Na₂CO₃, 0° C.; d) MnO₂, THF, RT; e) (MeO)₃CH, MeOH, p-TsOH, RT.

FIG. 10 shows a scheme for the synthesis of compound 19, a) 2-chloro-4-nitrophenol, TBABr, DCM, 0.75M NaOH; b) Et₃N, MeOH, H₂O (2:2:1).

FIG. 11 shows the ¹H NMR (D₂O, 800 MHz) spectra of compound 19.

FIG. 12 shows UPLC chromatograms (fluorescence detection) of the samples prepared as described in Example 10 in order to investigate the specificity of starch hydrolytic enzymes present in barley malt extract towards compound 19.

FIG. 13 shows the initial velocity (V₀) in the hydrolysis of 19 at 10-50% barley malt extract ([S]=0.5 mM).

FIG. 16 shows the Michaelis-Menten kinetics for barley malt extract when acting on compound 19, Lineweaver-Burk plot inserted in the lower panel.

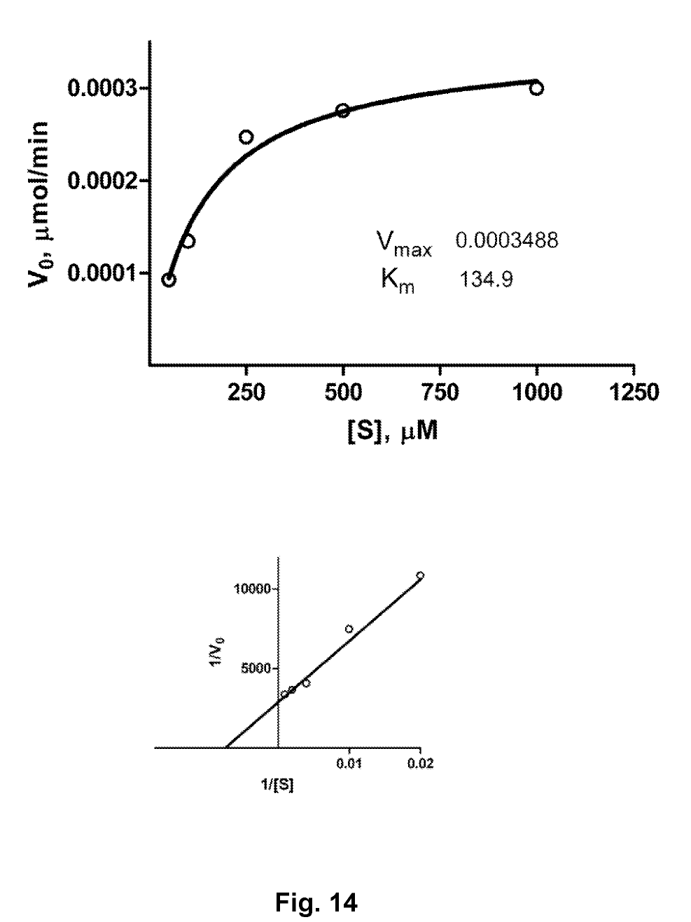

### DETAILED DESCRIPTION OF THE INVENTION

### Assay for Detecting α-(1→6)-Glucosidase Activity

The present invention relates to a method for detecting α-(1→6)-glucosidase activity in a sample. Said α-(1→6)-glucosidase activity may for example be pullulanase activity or it may be Limit Dextrinase activity. Thus, preferably, the invention relates to a method for detecting Limit Dextrinase activity in a sample. The sample may be any sample, such as any of the samples described herein below in the section “Sample”.

The method is useful for detecting α-(1→6)-glucosidase activity in a sample. The method is in particular useful for specifically detecting Limit Dextrinase activity, and it is one advantage of the method, that said activity may be determined in real time.

The method comprises incubation of an oligosaccharide substrate, which may be any of the oligosaccharide substrates described herein below in the section “Oligosaccharide substrate” together with said sample. In addition the method may comprise contacting the oligosaccharide substrate with an exo-glucosidase, which may be any of the exo-glucosidases mentioned herein below in the section “Exo-glucosidase”.

According to the invention the oligosaccharide substrate may be incubated with the sample and the exo-glucosidase simultaneously or sequentially. In embodiments where incubation is sequential the oligosaccharide substrate is in general first incubated with the sample and subsequently with the exo-glucosidase. It is however preferred that the oligosaccharide substrate is incubated with the sample and the exo-glucosidase simultaneously.

In addition to the sample and the oligosaccharide substrate, the incubation may optionally comprise one or more additional reagents. Said additional reagents may for example be selected from the group consisting of buffers, salts and detergents and reducing agents.

The buffer may be any useful buffer. It is frequently desirable that the incubation is done under slightly acidic conditions, and accordingly it is preferred that the buffer is capable of buffering to a slightly acidic pH. For example the incubation may be done at a pH in the range of 4 to 8, such as a pH in the range of 4.5 to 6.5, for example a pH in the range of 5 to 6. Accordingly, the buffer may preferably be a buffer capable of buffering a solution to any of the aforementioned pH ranges. Non-limiting examples of useful buffers include maleate buffer and acetate buffer.

The salt may be any useful salt, e.g. NaCl. Similarly, the detergent may be any useful detergent such as triton, e.g. triton-X100.

In particular, the sample and the oligosaccharide substrate may be incubated in the presence of a reducing agent. Said reducing agent may for example be dithiothreitol (DTT). It is preferred that the sample and the oligosaccharide substrate are incubated in the presence of a reducing agent in embodiments of the invention where the sample comprises a limit dextrinase inhibitor, such as barley limit dextrinase inhibitor (LDI). Thus, it is preferred that the sample and the oligosaccharide substrate are incubated in the presence of a reducing agent in embodiments of the invention, wherein the sample comprises a cereal or an extract thereof. Said reducing agent may be present in any suitable amount, preferably in an amount sufficient to reduce the disulphide bridges of a limit dextrinase inhibitor, such as the barley limit dextrinase inhibitor. Thus, for example if the reducing agent is DTT, the DTT may be present in a concentration of at least 10 mM, such as at least 20 mM, for example in the range of 20 to 100 mM.

The incubation may be done at any temperature, where the α-(1→6)-glucosidase is active, for example at any temperature where the Limit Dextrinase enzyme is active. In general the incubation is done at room temperature or higher. Thus, the incubation may for example be done at a temperature in the range of 20 to 65° C., such as at a temperature in the range of 30 to 60° C., for example at a temperature in the range of 35 to 55° C., such as at a temperature in the range of 40 to 50° C.

The incubation may be done for any suitable amount of time. In general the incubation should be at least 5 min, preferably at least 10 min, however it may also be longer. Thus, the incubation may be allowed to proceed for in the range of 10 min. to 3 hours, such as for in the range of 15 min. to 2 hours, for example for in the range of 0.5 to 1.5 hours.

After incubation the α-(1→6)-glucosidase activity, for example the Limit Dextrinase activity may be detected by determining the presence of semi-free detectable label or the level of free detectable label by any of the method described herein below in the section “Determining free detectable label”.

### Sample

The sample may be any sample in which it is desirable to known whether said sample contains α-(1→6)-glucosidase activity, and in particular the sample may be any sample in which it is desirable to known whether said sample contains Limit Dextrinase activity. Such samples may for example be samples obtained from cereals or other plants. Preferably the sample is obtained from cereals.

In particular, the presence of Limit Dextrinase is of major importance in order to achieve a high degree of fermentation in the brewing process. Accordingly, in preferred embodiments of the invention, the sample is derived from at least one ingredient for beverage production, for example for beer production. More preferably the sample comprises at least one ingredient for beverage production, for example beer production or an extract thereof. Alternatively, it is also preferred that the sample comprises an intermediate used during beverage production, for example during beer production or an extract thereof.

Thus, more preferably, the sample comprises a cereal or an extract thereof, wherein the cereal for example may be selected from the group consisting of barley, wheat, rye, oat, maize, rice, sorghum, millet, triticale, buckwheat, fonio and quinona. More preferably, the cereal is selected from the groups consisting of barley, wheat, rye, oat, maize and rice, more preferably the cereal is barley.

It is also a preferred embodiment of the invention that the sample comprises a malted cereal or an extract thereof, wherein the cereal for example may be selected from the group consisting of barley, wheat, rye, oat, maize, rice, sorghum, millet, triticale, buckwheat, fonio and quinona. More preferably, the cereal is selected from the groups consisting of barley, wheat, rye, oat, maize and rice, more preferably the cereal is barley.

Accordingly, in a very preferred embodiment of the invention the sample is malted barley or an extract thereof. Malted barley is also referred to as “barley malt” herein.

The term “malted” as used herein refers to that said cereal, e.g. said barley has been steeped and allowed to germinate, whereafter said steeped and germinated cereal has been dried, usually by kiln drying at elevated temperatures.

The sample may also be green malt from any of the above-mentioned cereals, or an extract thereof. Thus, the sample may be green malt of barley or an extract thereof. The term “green malt” as used herein denoted cereals, which have been steeped and allowed to germinate, but which have not undergone kiln drying or other treatment at elevated temperatures, e.g. temperatures above 50° C.

As outlined above, the sample may be an extract of a cereal and/or of malt and/or of green malt. The extract may in preferred embodiments be an aqueous extract. The extract may be preferred in any suitable manner, which results in an aqueous extract comprising enzymes from the cereal and/or malt. In general said extracts are prepared by grinding said cereal or said malt and incubating the ground cereal and/or malt with water optionally in the presence of one or more additional reagents. The cereal or the malt may be ground using any useful method, e.g. the cereal or the malt can be milled using a mill. The additional reagents may for example be salts, buffers or reducing agents. The reducing agent may for example be DTT. Extraction is in general performed at conditions, which will not inhibit or destroy the activity of α-(1→6)-glucosidase, and in particular the extraction is in general performed under conditions, which will not inhibit or destroy the activity of Limit Dextrinase. The temperature may thus be in the range from 0 to 50° C., such as in the range of 25 to 45° C. The extraction may be performed for example for in the range of 1 to 100 hours, such as for in the range of 5 to 50 hours, for example for in the range of 10 to 20 hours.

One non-limiting example of a useful method for preparing aqueous extracts of barley malt is described in example 2 herein below. The skilled person will understand that the same method can be employed for preparing aqueous extracts of other malts, as well as of unmalted cereals.

The sample may also comprise or consist of an intermediate obtained during production of a beverage, such as a cereal beverage. In particular, the sample may comprise or consist of an intermediate obtained during production of beer. Said intermediate may for example be wort. Said wort may be any wort, for example said wort may be selected from the group consisting of sweet wort, first wort, second wort, boiled wort and combinations thereof.

### Oligosaccharide Substrate

The invention in one aspect relates to an oligosaccharide substrate as well as to uses of said oligosaccharide substrate in methods for determining α-(1→6)-glucosidase activity and in particular in methods for determining limit dextrinase activity.

Said oligosaccharide substrate is preferably specific for limit dextrinase, meaning that the substrate preferably is cleaved by limit dextrinase much more efficiently than it is cleaved by any other glucosidase present in the sample. Thus, it is preferred that the substrate is cleaved at least twice, preferably at least 5 times, more preferably at least 10 times more efficiently by limit dextrinase than by β-amylase, α-amylase, α-glucosidase or β-glucosidase. In particular, it is preferred that the substrate is cleaved at least twice, preferably at least 5 times, more preferably at least 10 times more efficiently by limit dextrinase than by barley β-amylase, α-amylase or α-glucosidase. The efficiency is preferably determined by incubating the limit dextrinase or the other glucosidase with the oligosaccharide substrate and subsequently determining the level of the unreacted oligosaccharide substrate or the level of product from hydrolysis of the oligosaccharide substrate.

It is preferred that the oligosaccharide substrate comprises at least two glucoside residues linked by an α-(1→6)-glucosidic linkage. In particular, oligosaccharide portion of the oligosaccharide substrate should preferably be in the range of 4 to 10 sugars long and contain at least one α-(1→6) branch and contain at least one glucoside α-(1→4) bound to the glucoside on each side of the α-(1→6) branch

Thus, it is preferred that the oligosaccharide substrate is a compound of the formula I

X-(glucoside)ₙ-*(glucoside)ₘ-Z—Y

wherein
X is a blocking group capable of inhibiting cleavage by an exo-glucosidase of the bond between X and the adjacent glucoside; and
Y is a detectable label; and
Z is either S or O or N; and
-* is a α-(1→6)-glucosidic linkage; and
n and m individually are integers in the range of 1 to 6.

X may preferably be any of the blocking groups described herein below in the section “Blocking group” and Y may preferably be any of the detectable labels described in the section “Detectable label” herein below.

The term “glucoside” as used herein denotes glucose in the pyranose form (also denoted glucopyranose) covalently linked to another chemical moiety. Glucosides according to the present invention are in general bonded through their anomeric carbon to said other chemical moiety via a glycosidic bond. The glycosidic bond may be any glycosidic bond, but it is in general an O-glycosidic bond. Said glycosidic bond may be an α-glycosidic bond or a β-glycosidic bond.

It is preferred that the glucoside is a D-glucoside, and thus it is preferred that the glucoside is selected from the group consisting of α-D-glucopyranoside and β-D-glucopyranoside. It should be noted that the glucosides of the oligosaccharide substrate may all be similar, but they may also differ. Thus, it is preferred that each glucoside individually is selected from the group consisting of α-D-glucopyranoside and β-D-glucopyranoside.

It is generally preferred that all glucosides of the oligosaccharide substrate are α-D-glucopyranose except for the glucose at the reducing end, which may be either α-D-glucopyranoside or β-D-glucopyranoside.

The glucosides may be linked by any useful linkage, provided that the bond designated “-*” in the formula above is a α-(1→6)-glucosidic linkages. It is however preferred that all other glucosides are linked to each other through α-(1→4)-glucosidic linkages.

In one embodiment the oligosaccharide comprises 2 maltooligosaccharides covalenty linked to each other by a α-(1→6)-glucosidic linkages. Useful maltooligosaccharides may for example be selected from the group consisting of maltose, maltotriose, maltotetraose, maltopentaose, maltohexaose, maltoheptaose and maltooctaose.

n and m are individually integers in the range of 1 to 6. Thus, for example n may be an integer in the range of 2 to 6, such as in the range of 2 to 5, for example in the range of 2 to 4, such as in the range of 3 to 6, for example in the range of 3 to 5, such as in the range of 3 to 4, for example n may be 3. m may for example be an integer in the range of 1 to 6, for example in the range of 2 to 6, such as in the range of 2 to 5, for example in the range of 2 to 4, such as in the range of 3 to 6, for example in the range of 3 to 5, such as in the range of 3 to 4, for example m may be 3.

In one preferred embodiment of the invention the oligosaccharide substrate is a compound of the formula II or the formula III, wherein formula II is

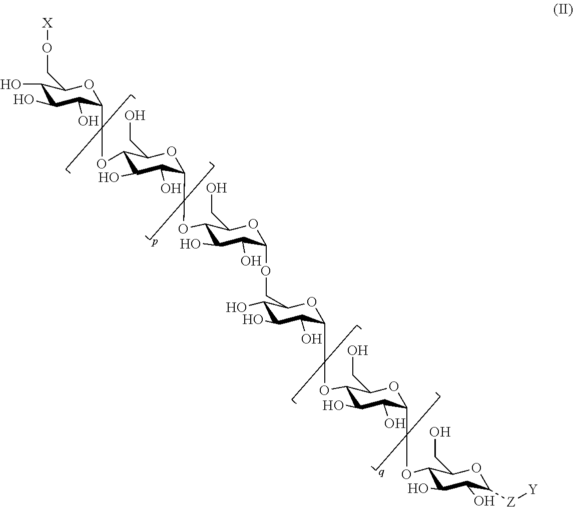

wherein
X is a blocking group capable of inhibiting cleavage by an exo-glucosidase of the bond between X and the adjacent glucoside; and
Y is a detectable label; and
Z is either N or O or S; and
p and q individually are integers in the range of 0 to 4; and
the dotted line indicates a bond, which may be either in the α or the β configuration and formula III is

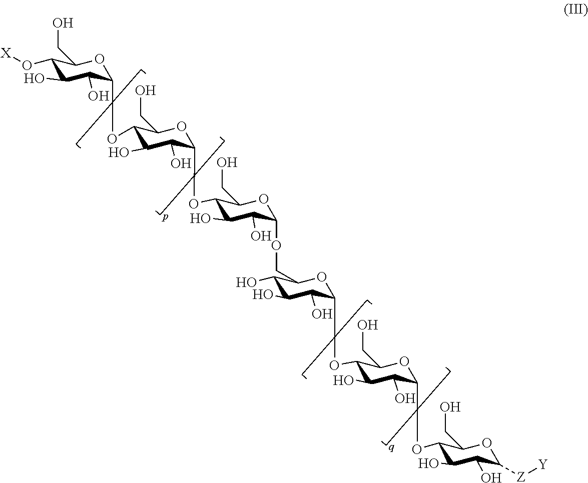

wherein
X is a blocking group capable of inhibiting cleavage by an exo-glucosidase of the bond between X and the adjacent glucoside; and
Y is a detectable label; and
Z is either N or S or O; and
p and q individually are integers in the range of 0 to 4; and
the dotted line indicates a bond, which may be either in the α or the β configuration.

It is preferred that the oligosaccharide substrate is a compound of formula II.

X in relation to compounds of formula II and III may preferably be any of the blocking groups described herein below in the section “Blocking group” and Y in relation to compounds of formula II or III may preferably be any of the detectable labels described in the section “Detectable label” herein below.

p and q are individually integers in the range of 0 to 4. Thus, for example p may be an integer in the range of 0 to 3, such as in the range of 0 to 2, for example in the range of 1 to 4, such as in the range of 1 to 3, for example in the range of 1 to 2, for example p may be 1. q may be an integer in the range of 0 to 3, such as in the range of 0 to 2, for example in the range of 1 to 4, such as in the range of 1 to 3, for example in the range of 1 to 2, for example q may be 1.

The dotted line preferably indicates an α-glucosidic bond or a β-glucosidic bond, more preferably the dotted line indicates an β-glucosidic bond.

Z may be N or S or O, however preferably Z is O.

Accordingly, in a preferred embodiment of the invention the oligosaccharide substrate is a compound of formula IV:

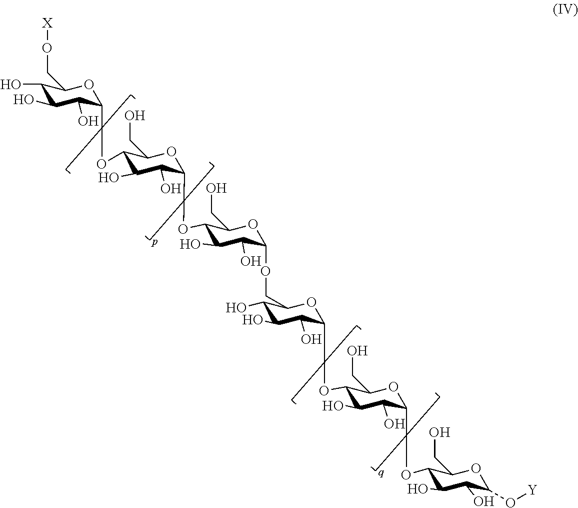

wherein
X is a blocking group capable of inhibiting cleavage by an exo-glucosidase; and
Y is a detectable label; and
p and q individually are integers in the range of 0 to 4; and
the dotted line indicates a bond, which may be either in the α or the β configuration.

X may preferably be any of the blocking groups described herein below in the section “Blocking group” and Y may preferably be any of the detectable labels described in the section “Detectable label” herein below.

In a very preferred embodiment of the invention the oligosaccharide substrate is a compound of formula V:

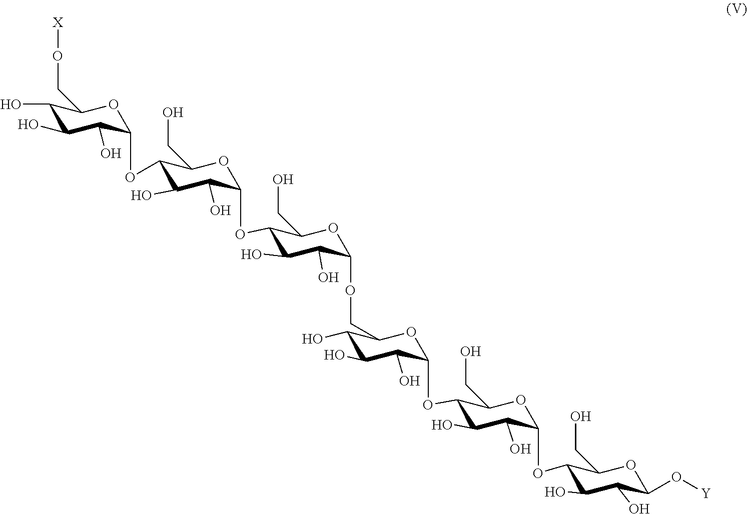

wherein X may be any of the blocking groups described herein below in the section “Blocking group” and Y may be any of the detectable labels described in the section “Detectable label” herein below.

### Blocking Group

The oligosaccharide substrate to be used with the present invention comprises a blocking group, which may also be denoted X. The blocking group according to the invention may be any group capable of inhibiting cleavage by an exo-glucosidase. Preferably, the blocking group is capable of inhibiting cleavage by an exo-glucosidase of the bond between X and an adjacent glucoside and/or of the bond between said adjacent glucoside and its neighbouring glucoside.

In particular, it is preferred that the blocking group (X) is capable of inhibiting cleavage by a glycoamylase of the bond between X and an adjacent glucoside.

In particular, it is preferred that the blocking group (X) is capable of inhibiting cleavage by an α-glucosidase of the bond between X and an adjacent glucoside.

In one embodiment it is furthermore preferred that the blocking group (X) is capable of inhibiting cleavage by a β-amylase of the bond between the glucoside adjacent to X and its neighbouring glucoside.

Moreover it is preferred that the blocking group (X) is capable of inhibiting cleavage by at least one glycoamylase, at least one α-glucosidase, and at least one β-amylase.

Yet more preferably, the blocking group (X) is capable of inhibiting cleavage by barley glycoamylase, barley α-glucosidase and barley β-amylase.

It is preferred that X is a glucoside linked to an adjacent glucoside by a glucosidic bond, wherein one or more —OH groups of said glucoside are substituted to yield —O—R, wherein R may be any group resulting in inhibition of cleavage of said glucosidic bond. Said —OH may be substituted with similar —O—R or with different —O—R groups.

The blocking group in general creates a terminal glucoside no longer capable of fitting the active site of the exo-enzyme, thereby blocking cleavage by that exo-enzyme. Thus the size and chemical composition of the blocking group in general are not critical, and many chemically diverse substituents are useful. Thus, virtually any substituent bonded to C2, C3, C4 or C6 of the glucoside part of X can block the action of exo-enzymes.

R may for example be selected from the group consisting of carboxylic acid esters (e.g. acetyl or benzoyl); phosphate esters; sulfonate esters (e.g. toluenesulfonyl or methanesulfonyl); ethers (e.g. methyl, benzyl, silyl and triphenylmethyl) and monosaccharides other than α-(1→4) linked glucose.

Alternatively, the blocking group can be an acetal or ketal blocking group, i.e. a group which blocks the C4 and C6 hydroxyls of the terminal glucose unit.

In one embodiment only one —OH group is substituted to yield —O—R₃, wherein R₃ preferably is as defined herein below. In another embodiment at least two —OH groups are substituted, wherein the two oxygen atom together with the two substituents form a ring structure. Said ring structure is preferably a 5 or 6 membered heterocycle containing 2 oxygen atoms and 3 to 4 carbon atoms.

In one preferred embodiment X has the structure

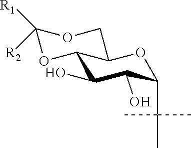

wherein
R₁ and R₂ individually are selected from the group consisting of —H, alkyl, aryl, heteroaryl and —COOH, wherein any of the aforementioned may optionally be substituted with one or more substituents, and
the dotted line indicates the point of attachment.

As used herein the term “alkyl” refers to a saturated, straight or branched hydrocarbon chain. The hydrocarbon chain preferably contains from one to six carbon atoms (C₁₋₆-alkyl), more preferred from one to three carbon atoms (C₁₋₃-alkyl), including methyl, ethyl, propyl and isopropyl.

The term “aryl” as used herein refers to a substituent consisting of a monocyclic or polycyclic aromatic hydrocarbon. Aryl according to the invention may for example be phenyl, tolyl, xylyl or napthyl. It is preferred that the aryl is a monocyclic aromatic hydrocarbon and more preferably aryl is phenyl.

The term “heteroaryl” as used herein refers to a substituent consisting of a monocyclic or polycyclic aromatic group containing one or more heteroatoms in the ring structure. The heteroatoms may preferably be selected from the group consisting of oxygen (O), nitrogen (N) and sulphur (S). It is preferred that the heteroaryl is a monocyclic aromatic group containing from one to two heteroatom(s) in the ring structure. More preferably the heteroaryl is a 5 to 6 membered monocyclic aromatic group containing from one to two heteroatoms in the ring structure. Thus heteroaryl may for example be selected from the group consisting of pyrrolyl, furanyl, thiophenyl, pyrazolyl, oxazolyl, thiazolyl, pyridyl, diazinyl and dioxinyl. In particular heteroaryl may be thiazolyl or diazinyl.

As used herein the term “substituent” in relation to organic compounds refers to a chemical group, which is attached to another chemical group by a covalently bond and which replaces an —H in said other chemical group.

As used herein the term “substituted with” in relation to organic compounds means that one —H has been replaced with the indicated group. By way of example, the term “glucoside substituted with —R” refers to glucoside, where one —OH has been replaced by —OR.

In one embodiment it is preferred that at the most one of R₁ and R₂ are —H. In another embodiment it is preferred that at least one of R₁ and R₂ is —H. Thus, in one embodiment R₂ is —H, whereas R₁ is from the group consisting of alkyl, aryl and heteroaryl, wherein any of the aforementioned may optionally be substituted with one or more substituents selected from the group consisting of alkyl, alkoxy and amino-alkyl.

The term “alkoxy” as used herein refers to an “alkyl-O-” group, wherein alkyl is as defined above.

The term “amino-alkyl” as used herein refers to -alkyl-NH₂, wherein alkyl is as defined above. Said —NH₂ group may be bound to any of the carbon atoms of the alkyl chain, for example to the terminal carbon.

R₁ and R₂ may individually be selected from the group consisting of —H, C₁₋₅-alkyl, aryl, heteroaryl and —COOH, wherein any of C₁₋₅-alkyl, aryl, heteroaryl may optionally be substituted with one or more substituents selected from the group consisting of C₁₋₅-alkyl, C₁₋₅-alkoxy and amino-C₁₋₅-alkyl.

In particular, R₁ and R₂ may individually be selected from the group consisting of —H, C₁₋₅-alkyl, 5 to 10 membered aryl, 5 to 10 membered heteroaryl and —COOH, wherein any of the aforementioned may optionally be substituted with one or more substituents selected from the group consisting of alkyl, alkoxy and amino-alkyl.

The term 5 to 10 membered aryl, refers to an aryl as defined herein above containing 5 to 10 atoms in its ring structure.

The term 5 to 10 membered heteroaryl, refers to a heteroaryl as defined herein above containing 5 to 10 atoms in its ring structure.

In a preferred embodiment R₁ and R₂ may individually be selected from the group consisting of —H, C₁₋₅-alkyl, 5 to 10 membered aryl, 5 to 10 membered heteroaryl and —COOH, wherein any of the aforementioned may optionally be substituted with one or more substituents selected from the group consisting of C₁₋₅-alkyl, C₁₋₅-alkoxy and amino-C₁₋₅-alkyl.

In a preferred embodiment R₁ and R₂ may individually be selected from the group consisting of —H, C₁₋₅-alkyl, 5 to 6 membered aryl and 5 to 6 membered heteroaryl, wherein any of the aforementioned may optionally be substituted with one or more substituents selected from the group consisting of C₁₋₃-alkyl, C₁₋₃-alkoxy and amino-C₁₋₃-alkyl.

In particular, R₁ and R₂ may individually be selected from the group consisting of —H, pyridyl and phenyl, wherein any of the aforementioned may optionally be substituted with one or more substituents selected from the group consisting of alkyl, alkoxy and amino-alkyl.

Thus, In particular, R₁ and R₂ may individually be selected from the group consisting of —H, pyridyl and phenyl, wherein any of the aforementioned may optionally be substituted with one or more substituents selected from the group consisting of C₁₋₆-alkyl, C₁₋₆-alkoxy and amino-C₁₋₆-alkyl.

Thus, In particular, R₁ and R₂ may individually be selected from the group consisting of —H, pyridyl and phenyl, wherein any of the aforementioned may optionally be substituted with one or more substituents selected from the group consisting of C₁₋₃-alkyl, C₁₋₃-alkoxy and amino-C₁₋₃-alkyl.

In one embodiment of the invention R₁ is —H and R₂ is selected from the group consisting of C₁₋₅-alkyl, 5 to 10 membered aryl and 5 to 10 membered aryl substituted with one or more C₁₋₅-alkyl.

In another embodiment R₁ is selected from the group consisting of C₁₋₅-alkyl, 5 to 10 membered aryl and 5 to 10 membered aryl substituted with one or more C₁₋₅-alkyl and R₂, independently, is selected from the group consisting of C₁₋₅-alkyl, 5 to 10 membered aryl and 5 to 10 membered aryl substituted with one or more C₁₋₅-alkyl and —COOH.

In another embodiment at least one of R₁ and R₂ is a quencher capable of quenching a fluorescent signal. This is in particular the case in embodiments of the invention, wherein Y consists of or comprises a fluorophore. It is preferred that said quencher is capable of quenching the fluorescence of said fluorophore.

It is also comprised within the present invention that R₁ may be selected from the group consisting of alkyl, aryl and heteroaryl, wherein any of the aforementioned is substituted with a quencher capable of quenching a fluorescent signal. R₁ may be selected from the group consisting of C₁₋₅-alkyl, 5 to 6 membered aryl and 5 to 6 membered heteroaryl, wherein any of the aforementioned is substituted with a quencher capable of quenching a fluorescent signal. If Y consists of or comprises a fluorophore, it is preferred that the quencher is capable of quenching the fluorescence of said fluorophore.

In yet another embodiment at least one of R₁ and R₂ is a fluorophore. This is in particular the case in embodiments of the invention, wherein Y consists of or comprises a quencher. It is preferred that said quencher is capable of quenching the fluorescence of said fluorophore.

It is also comprised within the present invention that R₁ may be selected from the group consisting of alkyl, aryl and heteroaryl, wherein any of the aforementioned is substituted with a fluorophore. R₁ may be selected from the group consisting of C₁₋₅-alkyl, 5 to 6 membered aryl and 5 to 6 membered heteroaryl, wherein any of the aforementioned is substituted with a fluorophore. If Y consists of or comprises a quencher, it is preferred that the quencher is capable of quenching the fluorescence of said fluorophore.

Useful quenchers and fluorophores are described in more detail herein below in the section “FRET”.

In another embodiment X has the structure

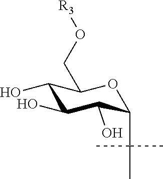

wherein R₃ is selected from the group consisting of alkyl, —(C═O)-alkyl, —(C═O)-aryl, —(C═O)-heteroaryl, aryl, heteroaryl, -alkyl-COOH, alkenyl, phosphate esters, sulfonate esters, silyl, quenchers, fluorophores and glycoside, wherein any of the aforementioned may optionally be substituted with one or more substituents and with the proviso that said glycoside is not a glucoside; and
the dotted line indicates the point of attachment.

As used herein the term “alkenyl” refers to an unsaturated, straight or branched hydrocarbon chain containing at least one double bond. It is preferred that the hydrocarbon chain contains only one double bond, more preferably the hydrocarbon chain contains one double bond and all other bonds are single bonds. The hydrocarbon chain preferably contains from one to six carbon atoms (C₁₋₆-alkenyl), more preferred from two to three carbon atoms (C₁₋₃-alkyl), including ethenyl, propenyl and isopropenyl.

In particular, R₃ may be selected from the group consisting of alkyl, —(C═O)-alkyl, —(C═O)-aryl, —(C═O)-heteroaryl, aryl, heteroaryl, -alkyl-COOH, alkenyl, phosphate esters, sulfonate esters and glycoside, wherein any of the aforementioned may optionally be substituted with one or more substituents selected from the group consisting of alkyl, alkenyl, aryl, heteroaryl and glycoside, with the proviso that said glycoside is not a glucoside.

In respect of R₃, then alkyl is preferably C₁₋₅ alkyl, such as C₁₋₃ alkyl. In particular, alkyl may be ethyl.

In respect of R₃ then alkenyl is preferably C₂₋₅-alkenyl. Alkenyl may in particular be selected from the group consisting of ketopropylidene, ketobutylidene and ethylidene.

In respect of R₃, then aryl is preferably a 5 to 10 membered aryl, more preferably a 5 to 6 membered aryl. In particular, the aryl may be phenyl.

R₃ may be aryl substituted with alkenyl. For example R₃ may be phenyl or pyridyl substituted with C₂₋₃-alkenyl.

In respect of R₃, then heteroaryl is preferably a 5 to 10 membered heteroaryl, more preferably a 5 to 6 membered heteroaryl. In particular, the aryl may be pyridyl.

R₃ may be alkyl substituted with one or more aryl, wherein alkyl and aryl preferably are as defined above. For example R₃ mat be triphenylmethyl.

R₃ may be —(C═O)-alkyl, wherein said alkyl may be as defined above. For example —(C═O)-alkyl may be ethylcarboxyl.

R₃ may be —(C═O)-aryl, wherein aryl may be as defined above. For example —(C═O)-aryl may be benzoyl.

R₃ may also be a sulfonate ester, for example toluenesulfonyl or methanesulfonyl.

R₃ may glycoside, with the proviso that said glycoside is not a glucoside. In particular, the glycoside may be any glycoside other than alpha-1,4 linked glucose. For example the glycoside may be β-D-galactopyranosyl.

In another embodiment R₃ is a quencher capable of quenching a fluorescent signal. This is in particular the case in embodiments of the invention, wherein Y consists of or comprises a fluorophore. It is preferred that said quencher is capable of quenching the fluorescence of said fluorophore.

In yet another embodiment R₃ is a fluorophore. This is in particular the case in embodiments of the invention, wherein Y consists of or comprises a quencher. It is preferred that said quencher is capable of quenching the fluorescence of said fluorophore.

In another embodiment X is —R₃,

wherein R₃ is selected from the group consisting of alkyl, —(C═O)-alkyl, —(C═O)-aryl, —(C═O)-heteroaryl, aryl, heteroaryl, -alkyl-COOH, alkenyl, phosphate esters, sulfonate esters, silyl, quenchers, fluorophores and glycoside, wherein any of the aforementioned may optionally be substituted with one or more substituents and with the proviso that said glycoside is not a glucoside.

In particular, R₃ may be selected from the group consisting of alkyl, —(C═O)-alkyl, —(C═O)-aryl, —(C═O)-heteroaryl, aryl, heteroaryl, -alkyl-COOH, alkenyl, phosphate esters, sulfonate esters and glycoside, wherein any of the aforementioned may optionally be substituted with one or more substituents selected from the group consisting of alkyl, alkenyl, aryl, heteroaryl and glycoside, with the proviso that said glycoside is not a glucoside.

In respect of R₃, then alkyl is preferably C₁₋₅ alkyl, such as C₁₋₃ alkyl. In particular, alkyl may be ethyl.

In respect of R₃ then alkenyl is preferably C₁₋₅-alkenyl. Alkenyl may in particular be selected from the group consisting of ketopropylidene, ketobutylidene and ethylidene.

In respect of R₃, then aryl is preferably a 5 to 10 membered aryl, more preferably a 5 to 6 membered aryl. In particular, the aryl may be phenyl.

R₃ may be aryl substituted with alkenyl. For example R₃ may be phenyl or pyridyl substituted with C₁₋₃-alkenyl. Thus R₃ may for example be benzylidene.

In respect of R₃, then heteroaryl is preferably a 5 to 10 membered heteroaryl, more preferably a 5 to 6 membered heteroaryl. In particular, the aryl may be pyridyl.

R₃ may be alkyl substituted with one or more aryl, wherein alkyl and aryl preferably are as defined above. For example R₃ mat be triphenylmethyl.

R₃ may be —(C═O)-alkyl, wherein said alkyl may be as defined above. For example —(C═O)-alkyl may be ethylcarboxyl.

R₃ may be —(C═O)-aryl, wherein aryl may be as defined above. For example —(C═O)-aryl may be benzoyl.

R₃ may also be a sulfonate ester, for example toluenesulfonyl or methanesulfonyl.

R₃ may glycoside, with the proviso that said glycoside is not a glucoside. In particular, the glycoside may be any glycoside other than alpha-1,4 linked glucose. For example the glycoside may be β-D-galactopyranosyl.

In another embodiment R₃ is a quencher capable of quenching a fluorescent signal. This is in particular the case in embodiments of the invention, wherein Y consists of or comprises a fluorophore. It is preferred that said quencher is capable of quenching the fluorescence of said fluorophore.

In yet another embodiment R₃ is a fluorophore. This is in particular the case in embodiments of the invention, wherein Y consists of or comprises a quencher. It is preferred that said quencher is capable of quenching the fluorescence of said fluorophore.

In yet another embodiment of the invention X is a glucoside, wherein said glucoside is linked to the adjacent glucoside by an α-(1→6) glucosidic linkage. When X is a glucoside linked to the adjacent glucoside by an one α-(1→6) glucosidic linkage, then said α-(1→6) glucosidic linkage cannot be cleaved by a number of exo-glucosidases. For example said α-(1→6) glucosidic linkage is not cleaved to any significant extent by exo-glucosidases normally found in barley malt extract. Accordingly, a glucoside linked to the adjacent glucoside by an α-(1→6) glucosidic linkage can be considered a “blocking group” within the meaning of the present invention.

In particular, in embodiments of the invention wherein the oligosaccharide substrate is a compound of formula III, IV or V, then X may be glucoside.

Thus, in one embodiment of the invention X has the structure

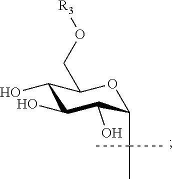

and wherein R₃ is —H, and wherein the dotted line indicates the point of attachment. Thus, in embodiments of the invention wherein the oligosaccharide substrate is a compound of formula III, IV or V, then X may have the structure

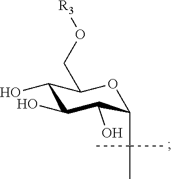

wherein R₃ is —H, and wherein the dotted line indicates the point of attachment.

### Detectable Label

The oligosaccharide substrate according to the present invention also comprises a detectable label, which may also be denoted Y.

The label may be any detectable label available to the skilled person, however in a preferred embodiment it is label, which is specifically detectable when the oligosaccharide substrate has been cleaved by a Limit Dextinase and optionally by one or more exoglucosidases. In general, the oligosaccharide substrate of the invention comprises a (1→6)-α-glucosidic linkage, which can be cleaved by Limit Dextrinase. Cleavage of the (1→6)-α-glucosidic linkage will free the next glucoside residue, so it can be cleaved of by an exo-glucosidase. This will eventually lead to liberation of Y in its free form. In embodiments of the invention wherein Z is O, then Y in its free form may be either in the form of Y—OH or Y—O⁻.

The term “Y—OH” refers to Y substituted with a hydroxyl group. The terms “Y—OH” and HO—Y″ are used interchangeably herein.

Thus, in a preferred embodiment Y is a detectable label, which may be differentially detected depending on whether Y is bound to the oligosaccharide substrate or whether Y is in its free form Y—OH. For example, Y may be a chromophore in its free form Y—OH, but colourless when bound to the oligosaccharide substrate. Alternatively, Y—OH may be a hapten, which is specifically recognised by an antibody, when in its free form, but not when it is bound to the oligosaccharide substrate. This will facilitate detection of whether the oligosaccharide substrate has been cleaved by Limit Dextrinase.

As used herein the term “hapten” refers to a small organic molecule that can induce formation of antibodies in vivo in a mammal, only when said hapten is attached to a large carrier e.g. a protein. Antibodies raised against said hapten-carrier adduct will be able to bind to the hapten even in the absence of the carrier.

In general, Y is linked to the reducing terminal glucoside of the oligosaccharide substrate by either an α-glucosidic bond or a β-glucosidic bond. Preferably, Y is bound to the reducing terminal glucoside of the oligosaccharide substrate by a β-glucosidic bond.

In preferred embodiments of the invention, where Z is O, then Y—OH may be a chromophore, a fluorophore, a chemiluminescent residue, a bioluminescent residues or a hapten. In particular, Y—OH may be selected from the group consisting of chromophores and haptens.

In a preferred embodiment of the invention Y—OH is a chromophore. In particular it is preferred that Y in its free form Y—OH is a chromophore, whereas Y when bound to the oligosaccharide substrate is not a chromophore. Preferably said chromophore is O-linked to said oligosaccharide substrate. Thus, it is preferred that when Y is a chromophore then Z is O. Upon hydrolysis of the bond between the reducing end glucoside and Y, then Y will be liberated in the form of Y—OH. In an aqueous solution Y—OH will typically be in equilibrium with Y—O⁻. It is preferred that Y—OH (or Y—O⁻) is a chromophore.

Thus, in a very preferred embodiment Y is nitrophenyl optionally substituted with one or more groups selected from the group consisting of hydroxyl, —(CH₂)ₜ—(C═O)—R₄ and halogen, wherein t is an integer in the range of 0 to 4 and R₄ is selected from the group consisting of alkoxy, —NH-alkyl, —NH₂ and —OH. t may be an integer in the range of 0 to 4, such as in the range of 0 to 2, however preferably t is 1. Thus Y may be nitrophenyl, for example 2-nitrophenyl or 4-nitrophenyl substituted with one or more groups selected from the group consisting of hydroxyl, —(CH₂)—(C═O)—R₄ and halogen, wherein R₄ is selected from the group consisting of C₁₋₃-alkoxy, —NH—C₁₋₃-alkyl, —NH₂ and —OH. In particular, Y may be nitrophenyl, for example 2-nitrophenyl or 4-nitrophenyl substituted with one or more groups selected from the group consisting of hydroxyl and halogen. Said halogen may preferably be selected from the group consisting of chloro, flouro and bromo, more preferably the halogen is selected from the group consisting of fluoro and chloro.

Thus, Y may be a nitrophenyl residue, for example Y may be selected from the group consisting of 4-nitrophenyl, 2-chloro-nitrophenyl, 2-chloro-4-nitrophenyl, 2,6-dichloro-4-nitrophenyl, 2-fluoro-4-nitrophenyl, 2,6-difluoro-4-nitrophenyl, 2-bromo-4-nitrophenyl, 2,6-dibromo-4-nitrophenyl, 2-nitrophenyl, 2-hydroxy-4-nitrophenyl, 3-hydroxy-4-nitrophenyl, 3-nitrophenyl, 2,4-di-nitrophenyl, 4-chloro-2-nitrophenyl and nitrophenylacetic acid esters or amides. In particular, Y may be selected from the group consisting of p-nitrophenol, o-nitrophenyl, m-nitrophenyl, p-nitrophenyl, 2,4-nitrophenyl, 4-chloro-2-nitrophenyl or 4-hydroxy-3-nitrophenylacetic acid esters or amides.

Said 4-hydroxy-3-nitrophenylacetic acid esters or amides are preferably selected from the group consisting of methyl-4-hydroxy-3-nitrophenylacetate, ethyl-4-hydroxy-3-nitrophenylacetate, amino-4-hydroxy-3-nitrophenylacetate, amino-methyl-4-hydroxy-3-nitrophenylacetate and amino-ethyl-4-hydroxy-3-nitrophenylacetate.

Y may also be a chemiluminescent residue, for example Y may be luciferin

In one embodiment of the invention Y is a fluorophore. In this embodiment of the invention it is preferred that X comprises a quencher capable of quenching the fluorescence of Y. Due to the quencher the oligosaccharide substrate will not emit fluorescence, but upon cleavage of the (1→6)-α-glucosidic linkage, the quencher and the fluorophore can move apart, and thereby the activity of the Limit Dextrinase can be monitored.

Fluorophores may for example be selected from the group consisting of TAMRA, resorufin and coumarin derivatives such as umbelliferyl, 4-methylumbelliferyl or 4-trifluoromethylumbelliferyl.

In another embodiment of the invention Y may be a quencher. In this embodiment of the invention it is preferred that X comprises a fluorophore and said quencher is capable of quenching the fluorescence of said fluorophore. Due to the quencher the oligosaccharide substrate will not emit fluorescence, but upon cleavage of the α-(1→6)-glucosidic linkage, the quencher and the fluorophore can move apart, and thereby the activity of the Limit Dextrinase can be monitored.

In embodiments of the invention, where Y is a fluorophore Z may be selected from the group consisting of O, S and N.

Useful quenchers and fluorophores are described in more detail herein below in the section “FRET”.

In another embodiment of the invention Y may be a hapten. More preferably Y—OH is a hapten. In particular, Y—OH may be a hapten, which is specifically recognised by a antibody when in its free form, but which is not recognised by said antibody when bound to the oligosaccharide substrate. The hapten Y—OH may also be a hapten which can be detected by an antibody when it is conjugated or attached to a protein or conjugated or attached to a surface.

In another embodiment Y may be selected from the group consisting of phenyl, 1-naphthyl, 2-methylphenyl, 2-methyl-1-naphtyl, 2-chlorophenyl, 4-chlorophenyl, 2,6-dichlorophenyl, 2-methoxyphenyl, 4-methoxyphenyl, 2-carboxyphenyl, 2-sulfophenyl, 2-sulfo-1-naphthyl, 2-carboxy-1-naphtyl, indoxyl, 5-bromoindoxyl and 4-chloro-3-bromoindoxyl.

In one preferred embodiment Y is selected from the group consisting of umbelliferyl, 4-methyl umbelliferyl, 1-naphtyl, o-nitrophenyl, m-nitrophenyl, p-nitrophenyl, 2,4-nitrophenyl, 4-chloro-2-nitrophenyl or 4-hydroxy-3-nitrophenylacetic acid esters or amides, wherein said esters and amides preferably are as defined herein above.

### FRET

In one embodiment of the invention X comprises a quencher and Y comprises or consists of a fluorophore. In another embodiment of the invention X comprises a fluorophore and Y comprises or consists of a quencher. Preferably, said quencher and said fluorophore are capable of FRET.

FRET is traditionally used for monitoring the distance between a fluorophore (also known as donor) and quencher (also known as acceptor) as FRET can occur if a donor and an acceptor are within a distance of 10-100 Å. Furthermore the absorption spectrum of the acceptor should overlap with the fluorescence emission spectrum of the donor. As long as an oligosaccharide substrate containing a donor and an acceptor is intact, FRET will occur and the fluorescence of the donor will be quenched by the acceptor. When the oligosaccharide substrate is cleaved by Limit dextrinase the donor and acceptor become separated, FRET will no longer occur resulting in increasing fluorescence at the emission wavelength of the donor.

Thus, in one embodiment Y comprises or consists of a fluorophore, and X comprises a quencher, wherein the emission spectrum of the fluorophore has an overlap with the excitation spectrum of the quencher. In another embodiment X comprises or consists of a fluorophore, and Y comprises or consists of a quencher, wherein the emission spectrum of the fluorophore has an overlap with the excitation spectrum of the quencher.

Non-limiting examples of useful FRET donor and acceptor pairs includes TAMRA and BHQ-1, TAMRA and BHQ-2, EDANS and DABCYL, Cy3 and Cy5 or fluorescein and TAMRA. Accordingly, X may comprise one part of each of the aforementioned FRET pairs, whereas Y may comprise or consist of the other part of the pair.

### Method for Preparing Oligosaccharide Substrates

The oligosaccharide substrates according to the invention may be prepared by any useful method known to the skilled person.

The oligosaccharide portion of the substrate may be obtained commercially, e.g. from Megazyme, Ireland. Alternatively, the oligosaccharide portion of the substrate may be prepared from a polysaccharide by enzymatic degradation. Said polysaccharide may for example be selected from the group consisting of amylopectin, pullulan or branched cyclodextrins.

The oligosaccharide portion of the substrate may also be synthesised chemically by standard carbohydrate coupling chemistry for example by carbohydrate coupling chemistry using protected donors and acceptors as described in Handbook of Chemical Glycosylation, edited by Alexei V. Demchenko, 2008, Wiley-VCH.

The label Y is usually attached to the reducing end glucoside. Y may be attached to the reducing end glucoside by standard glycosylation methods such as e.g. by biphasic glycosylation described in Handbook of Chemical Glycosylation, edited by Alexei V. Demchenko, 2008, Wiley-VCH.

When X comprises or consists of a substituted glucoside, X may be directly introduced onto the nonreducing glucose by standard methods of glycosylation, for example chemically as well as enzymatically. X may alternatively be formed by chemical modification of the already existing oligosaccharide by e.g. selective benzylidene formation or selective alkylation of the non-reducing glucose.

### Determining Semi-Free and Free Detectable Label

The methods of the invention comprise a step of incubating an oligosaccharide substrate according to the invention with a sample. If α-(1→6)-glucosidase activity is present and in particular if Limit dextrinase is present in said sample this will result in cleavage of the α-(1→6)-glucosidic linkage of the oligosaccharide substrate. This will result in liberation of the detectable label coupled to one or more glucosides. In general, after cleavage catalysed by α-(1→6)-glucosidase, for example by Limit Dextrinase then all of the glucosides attached to the detectable label will then be connected by (1→4)-glucosidic linkages. A detectable label connected to glucosides only connected by (1→4)-glucosidic linkages may also be referred to as a “semi-free detectable label” herein.

Preferably, the methods of the invention also comprise incubation of the oligosaccharide substrate with one or more exo-glucosidases. Due to the presence of the blocking group at the non-reducing end of the oligosaccharide substrate, then exo-glucosidases in general are not capable of cleaving the intact oligosaccharide substrate or they are only capable of cleaving the intact oligosaccharide substrate to a limited extent. However, after cleavage of the oligosaccharide substrate by a α-(1→6)-glucosidase, for example by Limit Dextrinase, the exo-glucosidase will be capable of cleaving off glucosides connected by (1→4)-glucosidic linkages resulting in liberation of the detectable label. Such liberated detectable label is also referred to as free detectable label herein.

The semi-free or the free detectable label may be detected by various means dependent on the nature of the detectable label.

In embodiments of the invention wherein the detectable label HO—Y is a hapten, then it is preferred that the hapten is detected using an antibody specifically recognising the free detectable label, i.e. the free hapten. Thus, it is preferred that the detectable label Y—OH is detected using an antibody, which binds to the free hapten. with a much higher affinity than to the hapten, when covalently linked to the oligosaccharide substrate. Said antibody may also be an antibody, which binds to the free hapten and/or to the hapten conjugated to a protein or a surface or to a group that can be attached to a protein or a surface. The antibody can be detected using any one of a number of conventional methods known to the skilled person. For example the antibody can be directly linked to a fluorophore or a chromophore allowing detection. The antibody can also be linked to an enzyme, such as peroxidase, which catalyses a reaction, which may be detected. The antibody may also be detected by a secondary antibody capable of binding to the first antibody, wherein the secondary antibody may be linked to a fluorophore, a chromophore or to an enzyme.

In embodiments of the invention where Y—OH is a chromophore, then it is preferred that the absorption wavelength of Y—OH is different depending on whether Y is attached to oligosaccharide substrate or whether Y is in the form of a free label Y—OH. Free detectable label, which is a chromophore may be detected by any conventional means for detecting chromophores for example by measuring absorbance at the relevant wavelength. The presence of the chromophore may also be determined by visual inspection.

In embodiments of the invention where Y is a fluorophore, then it is preferred that X comprises a quencher capable of quenching the fluorescence of said fluorophore. Presence of the semi-free detectable label or of the free detectable label may then be determined by measuring fluorescence using any suitable apparatus, such as a fluorescence plate reader.

### α-(1→6)-Glucosidase Activity

The methods of the invention may be used to determine α-(1→6)-glucosidase activity: said α-(1→6)-glucosidase may for example be the activity of a pullulanase or the activity of Limit Dextrinase. Thus the methods of the invention may be used to determine any activity of any Limit Dextrinase enzyme.

In embodiments of the invention wherein the sample is a plant, an extract of a plant or a plant product, then the methods are in particular useful for detecting the activity of Limit Dextrinase. Thus, when the sample is a cereal, an extract of a cereal or a product of a cereal, then the methods are in particular useful for detecting the activity of Limit Dextrinase. For example, when the sample is barley, an extract of barley or a barley based product, then the methods are in particular useful for detecting the activity of Limit Dextrinase.

Pullulanase is preferably an enzyme capable of catalyzing hydrolysis of α-(1→6)-D-glucosidic linkages in pullulan, amylopectin and glycogen. Pullulan is a linear polymer of 1→6-linked maltotriose units. Pullulanase according to the present invention are preferably pullulanases classified under EC 3.2.1.41.

Limit dextrinase according to the invention is a starch debranching enzyme, catalyzing cleavage of α-(1→6)-glycosidic bonds in branched dextrins. In particular Limit Dextrinases according to the present invention are Limit Dextrinase enzymes classified under E.C. 3.2.1.142

The Limit Dextrinase may for example be barley Limit Dextrinase, such as the protein of SEQ ID NO: 1.

Thus the Limit Dextrinase may be Limit Dextrinase of SEQ ID NO:1 or a functional homologue sharing at least 70%, preferably at least 80%, more preferably at least 85%, such as at least 90%, for example at least 95%, such as at least 98% sequence identity with SEQ ID NO:1. The sequence identity should be determined over the entire length of SEQ ID NO:1. A functional homologue of SEQ ID NO:1 is a protein, which is capable of catalyzing cleavage of α-(1→6)-glycosidic bonds in branched dextrins.

### Exo-Glucosidase

The methods of the invention optionally contain a step of incubation with one or more exo-glucosidase(s). In embodiments of the invention where the detection requires or preferably is done by detecting free detectable label, then it is preferred that the methods comprise a step of incubation with one or more exo-glucosidase(s).

The exo-glucosidase may be any exo-glucosidase, but preferably the exo-glucosidase is an enzyme capable of catalysing cleavage of terminal (1→4)-glucosidic linkages.

In particular the exo-glucosidase may be selected from the group consisting of glucan 1,4-α-glucosidase, α-glucosidase and β-glucosidase.

The α-glucosidase to be used in the present invention, is an enzyme capable of catalysing hydrolysis of terminal, non-reducing end (1→4)-linked α-D-glucose residues resulting in release of α-D-glucose. The α-glucosidase may thus in particular be α-D-glucoside glucohydrolase, The α-glucosidase is preferably an enzyme classified under EC.3.2.1.20. The α-glucosidase may be of any origin for example it may be of animal, plant or microbial origin. The α-glucosidase may also be prepared by recombinant technology. In particular, the α-glucosidase may be α-glucosidase of a Bacillus spp, preferably of Geobacillus stearothermophilus. α-glucosidase of Geobacillus stearothermophilus is commercial available from e.g. Megazyme, Ireland.

Thus the α-glucosidase may be α-glucosidase of SEQ ID NO:2 or a functional homologue sharing at least 70%, preferably at least 80%, more preferably at least 85%, such as at least 90%, for example at least 95%, such as at least 98% sequence identity with SEQ ID NO:2. The sequence identity should be determined over the entire length of SEQ ID NO:2. A functional homologue of SEQ ID NO:2 is a protein, which is capable of catalysing hydrolysis of terminal, non-reducing end (1→4)-linked α-D-glucose residues resulting in release of α-D-glucose.

In general it is preferred that at least one exo-glucosidase is α-glucosidase, when one or more of the glucosides positioned from the (1→6)-α-glucosidic linkage to the reducing end of the oligosaccharide substrate are connected by (1→4)-α-glucosidic linkages.

The exo-glucosidase may also be β-glucosidase. The β-glucosidase may be used together with an α-glucosidase or it may be used alone. In general it is preferred that at least one exo-glucosidase is β-glucosidase, when the oligosaccharide substrate contains a β-glucosidic linkage at the reducing end.

The β-glucosidase to be used in the present invention, is an enzyme capable of catalysing hydrolysis of terminal, non-reducing end β-D-glucosyl residues resulting in release of reducing D-glucose. The β-glucosidase is preferably an enzyme classified under EC.3.2.1.21. The β-glucosidase may be of any origin for example it may be of animal, plant or microbial origin. The β-glucosidase may also be prepared by recombinant technology. In particular, the β-glucosidase may be from the β-glucosidase of Agrobacterium spp. β-glucosidase of Agrobacterium is commercial available from e.g. Megazyme, Ireland.

Thus the β-glucosidase may be β-glucosidase of SEQ ID NO:3 or a functional homologue sharing at least 70%, preferably at least 80%, more preferably at least 85%, such as at least 90%, for example at least 95%, such as at least 98% sequence identity with SEQ ID NO:3. The sequence identity should be determined over the entire length of SEQ ID NO:3. A functional homologue of SEQ ID NO:3 is a protein, which is capable of catalysing hydrolysis of terminal, non-reducing end β-D-glucosyl residues resulting in release of β-D-glucose.

The glucan 1,4-α-glucosidase to be used in the present invention, is an enzyme capable of catalysing hydrolysis of terminal (1→4)-linked α-D-glucose residues successively from non-reducing ends of the chains resulting in release of reducing D-glucose. The glycoamylase is preferably an enzyme classified under E.C.3.2.1.3. Glucan 1,4-α-glucosidase may also be designated glycoamylase. The glucan 1,4-α-glucosidase may be of any origin for example it may be of animal, plant or microbial origin.

In one embodiment of the invention the exo-glucosidase may be β-amylase. According to the invention β-amylase is an enzyme capable of catalysing hydrolysis of α-(1→4)-D-glucosidic linkages in polysaccharides so as to remove successive maltose units from the non-reducing ends. Preferably, the β-amylase is an enzyme classified under EC 3.2.1.2.

The methods may comprise incubation with only one exo-glucosidase or incubation with several different exo-glucosidases, e.g. with 2, such as with 3, for example with 4 different exo-glucosidases. If more than one exo-glucosidase is used, the incubation with the different exo-glucosidases may be simultaneous or sequential dependent, however preferably it is simultaneous. It is preferred to use more than one exo-glucosidase, in particular to use 2 exoglucosidases. In one preferred embodiment the methods comprises incubation of the oligosaccharide substrate with α-glucosidase and β-glucosidase.

### SEQUENCE LISTING

| | |
|---|---|
| SEQ ID NO: 1 | Protein sequence of limit dextrinase of Hordeum |
| | vulgare subsp. vulgare |
| SEQ ID NO: 2 | Protein sequence of exo-alpha-1,4-glucosidase from |
| | Geobacillus stearothermophilus |
| SEQ ID NO: 3 | Protein sequence of beta-glucosidase from |
| | Agrobacterium sp. H13-3. |

### EXAMPLES

The invention is further illustrated by the following examples, which however should not be construed as being limiting for the invention.

All commercial available solvents and reagents were used without further purification. Organic solvents were removed under diminished pressure at <40° C. (bath temperature). TLC was carried out on silica plates (Merck 60 F₂₅₄ aluminium plates) with detection by UV-light (short wavelength) or 10% sulphuric acid in ethanol. Column chromatography was performed on silica gel (Merck, 230-400, 60 Å) and reversed phase chromatography was performed on Sep-pak plus C18 cartridge. Preparative TLC was performed by applying the sample to a Whatman prep. TLC plate, Partisil, PLK5F, silicagel 150 Å, 20 times 20 cm, 1000 μm and eluting with the mentioned eluent. ESI-MS was recorded on a Bruker Esquire 3000-Plus Ion Trap instrument with samples injected as solutions in 1:1 MeCN-water mixtures. HR-ESI-MS was recorded on a Q-Tof Ultima instrument from Micromass with appropriate internal standards used for lock mass. MALDI-ToF-MS was performed on a Bruker Daltonics Microflex instrument operating in reflectron mode. A 340 nm laser was used and mass spectra were typically accumulated from 100 laser shots. NMR spectra were recorded on a Bruker Avance III 400 MHz spectrometer operating at 300 K. Spectra are internally referenced to the solvent residue. Spectra were processed using Bruker Topspin 2.0. UPLC analysis was performed on Waters Acquity System. Equipped with TUV Detector (Dual wavelength UV detector), FLR detector (fluorescence detector), SQ-Detector (Single quadropole ESI-MS detector), Binary Solvent Manager, Sample Manager and Column Oven Manger. Separation was performed on reverse phase column (Waters ACQUITY BEH C18, 1.7 μm, 50 mm×2.1 mm) maintained at 26.0° C. by temperature control module (Column Oven Manager).

### Example 1

### Synthesis of Substrate 6

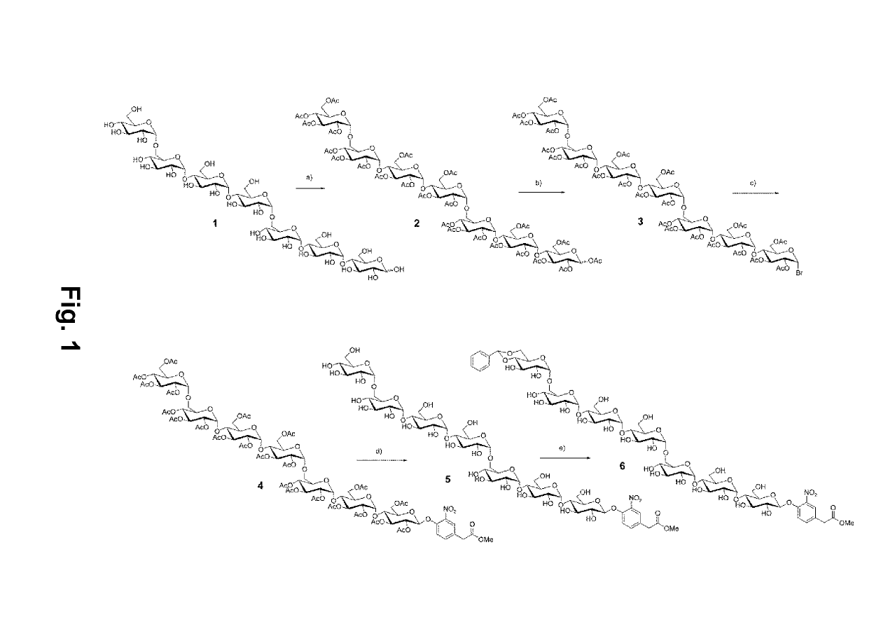

The example describes synthesis of an example of an oligosaccharide substrate designated compound 6. FIG. 1 shows a scheme of the synthesis as well as the chemical structure of compound 6. All compound numbers indicated in this example refers to the numbers indicated in FIG. 1.

### A: Synthesis of Compound 2

Glc-maltotriosyl-maltotriose 1 (Megazyme) (34.2 mg, 0.03 mmol) was suspended in dry pyridine (5 ml). Acetic anhydride (5 mL) and a catalytic amount of DMAP was added and the mixture was left at room temperature over night. The solvents were evaporated in vacuo. The residue was dissolved in DCM, washed with aqueous HCl (0.2 M), sat. NaHCO₃, brine, dried (Na₂SO₄), filtered and evaporated in vacuo. This gave a quantitative amount of compound 2 as a slightly orange solid. Used in the next step without further purification.

### B. Synthesis of Compound 3

Compound 2 (112 mg, 0.05 mmol) was dissolved in dichloromethane (3 ml). Acetic anhydride (0.5 mL) was added together with a few drops of HBr (33% in AcOH). The mixture was stirred at room temperature for 20 min. HBr (33% in AcOH, 3 mL) was added and the mixture stirred at room temperature for 2 hours. The mixture was evaporated in vacuo, dissolved in dichloromethane and washed with ice-cold saturated NaHCO₃, dried (Na₂SO₄), filtered and evaporated in vacuo to give compound 3 as a solid (60 mg, 53%). This was used in the next step without further purification.

### C. Synthesis of Compound 4

Compound 3, (63 mg, 0.03 mmol), 4-hydroxy-3-nitrophenylacetic acid methyl ester (59 mg, 0.28 mmol) and Tetrabutylammonium bromide (40 mg, 0.12 mmol) was dissolved in dichloromethane (3 mL) and NaHCO₃ (1 M, 2 mL) and KCl (1M, 1 mL). The mixture was heated under Argon at 45° C. over night. Extra dichloromethane was added and the organic phase was extracted with saturated NaHCO₃, brine, dried (Na₂SO₄), filtered and evaporated in vacuo. The residue was purified by flash chromatography (EtOAc/hexane, 85:15) to give a colourless solid (34 mg, 51%), containing a minor amount of the glucal (elimination product) as well as desired compound. This was used in the next step without further purification.

### D. Synthesis of Compound 5

Compound 4 (32 mg, 0.014 mmol) was dissolved in dry MeOH (4 ml). NaOMe (1 M, 10 μL) was added and the mixture stirred over night at room temperature. The mixture was neutralised with amberlite IR120 H⁺, filtered and evaporated. The residue was dissolved in water and purified on a Sep-pak C18 cartridge (2×200 mg) with 100% water to 20% MeCN with increments of 2.5% MeCN to give compound 5 as a white solid (13.5 mg, 71%). Pure judged from ¹H-NMR spectrum.

m/z [M+Na]: 1368.5

¹H-NMR (400 MHz, D₂O): δ 7.88 (s, 1H), 7.57 (d, J=8.8 Hz, 1H), 7.39 (d, J=8.7 Hz, 1H), 5.43 (d, J=3.5 Hz, 1H), 5.40-5.35 (m, 3H), 5.24 (d, J=7.5 Hz, 1H), 4.94 (m, 2H), 4.02-3.36 (m, 47H).

### E. Synthesis of Compound 6

Compound 5 (13.5 mg, 0.01 mmol) was dissolved in dry DMF (500 μL). Benzaldehyde dimethylacetal (10 μL, 0.066 mmol) was added followed by a cat. amount of camphor sulfonic acid. The mixture was stirred for 4 hours at 60° C. In all 30 μL extra benzaldehyde dimethylacetal was added with intervals. Et₃N (10 μL) was added and the compound was purified by preparative TLC, Whatman, Partisil PLK5F silicagel 150 Å, 20*20 cm, 1000 μm thickness, (MeCN/H₂O, 7.5/2.5). The band containing the desired compound was scraped off, extracted with MeCN/H₂O (3×5 mL, 3:1). The silica was removed by centrifugation and the residue was evaporated in vacuo. Purified further on Sep-pak C18 plus cartridge, eluting with H₂O/MeCN 5% increments (2 ml of each concentration). Lyophilisation gave compound 6 as a white solid (2.81 mg, 19.5%). Pure judged from ¹H-NMR spectrum.

m/z [M+Na]: 1456.5

¹H-NMR (400 MHz, D₂O): δ 7.89 (s, 1H), 7.59-7.39 (m, 7H), 5.74 (s, 1H), 5.44 (d, J=3.5 Hz, 1H), 5.41 (d, J=3.8 Hz, 1H), 5.36 (d, J=3.7 Hz, 1H), 5.33 (d, J=3.5 Hz, 1H), 5.25 (d, J=7.8 Hz, 1H), 5.01 (d, J=4.0 Hz, 1H), 4.94 (d, J=3.3 Hz, 1H), 4.28 (m, 1H), 4.06-3.40 (m, 47H).

### Example 2

### Limit Dextrinase Activity Assay

Compound 6 prepared as described in Example 1 was used to determine the activity of limit dextrinase in buffer and malt extract.

(1) For determining limit dextrinase activity, the following solutions were prepared.
(A) 50 mM sodium acetate buffer pH 5.5 supplemented with 200 mM NaCl and 0.005% (v/v) triton-X100.
(B) 240 μM compound 6 (prepared as described in Example 1), 480 mU/mL α-glucosidase from Bacillus stearothermophilis (Megazyme, Ireland), 480 mU/mL β-glucosidase from Agrobacterium sp. (Megazyme, Ireland) dissolved in (A).
(C) 250 nM recombinant barley limit dextrinase (produced according to Vester-Christensen et al., 2010) dissolved in (A).
(D) A sample of barley malt extract for determination of total limit dextrinase activity content therein was prepared by extracting 100 g milled barley malt in 300 mL nanopure water supplemented with 1.6 mg DTT (25 mM) on a magnetic stirrer at 40° C. for 12 hours. The extract was filtered (MN 614%, Macherey-Nagel) at 4° C. before use to remove husk and insoluble material.
(E) 2% (w/v) 2-Amino-2-hydroxymethyl-propane-1,3-diol (Trizma-Base, Sigma-Aldrich).

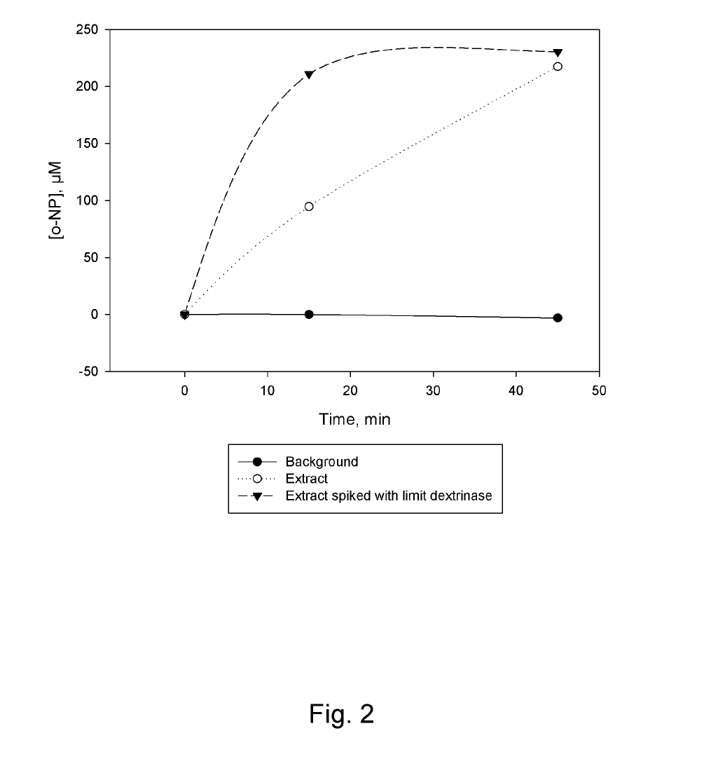

(2) The limit dextrinase activity was determined by the following procedure. After temperature equilibration to 50° C. in a water bath, 200 μL solution (B) was mixed with 50 μL (A), (C) or (D) and incubated at 50° C. At set time intervals 50 μL aliquots were taken out and mixed 1:1 with (E) to quench the limit dextrinase activity and develop the yellow color of o-nitrophenol (o-NP). The quenched reactions were kept on ice until determination of the absorbance at 430 nm. The concentration of o-NP in the samples were calculated using Lambert-Beer's law and a o-NP extinction coefficient of 4200 M⁻¹cm⁻¹. A bright yellow color accompanied a time dependent increase in absorbance at 430 nm of the samples containing limit dextrinase or extract. The control sample without limit dextrinase stayed colorless at all time points (FIG. 2).

### Comparative Example 2.1

### Limit Dextrinase Activity Assay in Malt Extract Inhibited with Limit Dextrinase Inhibitor

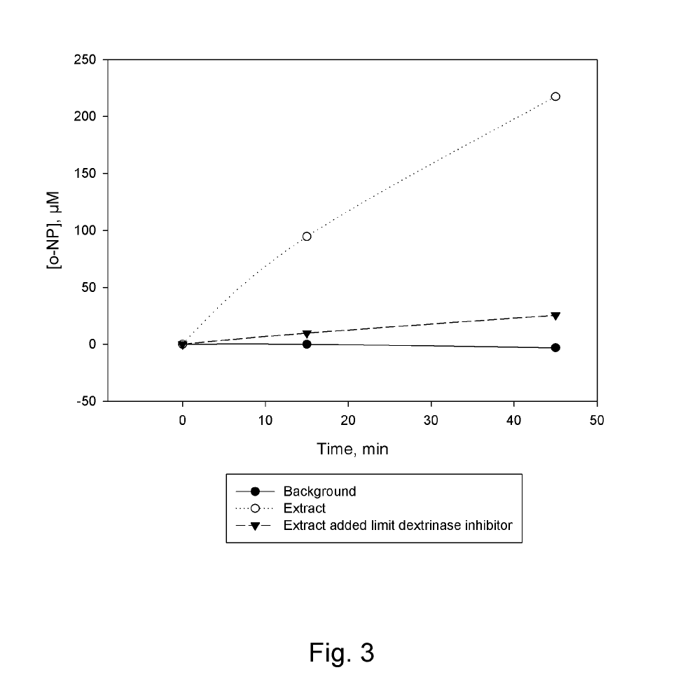

The procedure in Example 2 was repeated except that (C) was substituted with 50 μM limit dextrinase inhibitor (produced according to Jensen et al., 2011) dissolved in (B) and the reactions were started by mixing 200 uL of either (B) or (C) with 50 μL (D). In the sample added limit dextrinase inhibitor o-NP was released at 1/10 the rate of the uninhibited sample (FIG. 3). This results were comparable to determinations of limit dextrinase activity made using the commercially available limit dextrinase substrate limit dextrizyme (Megazyme, Ireland).

### Comparative Example 2.2

### α-Amylase Catalysed Hydrolysis of Compound 6

The procedure in Example 2 was repeated except that (D) was replaced by a 0-1750 mU dilution series of α-amylase from A. oryzae (Sigma-Aldrich) dissolved in (A) and the reactions were quenched after 30 min.

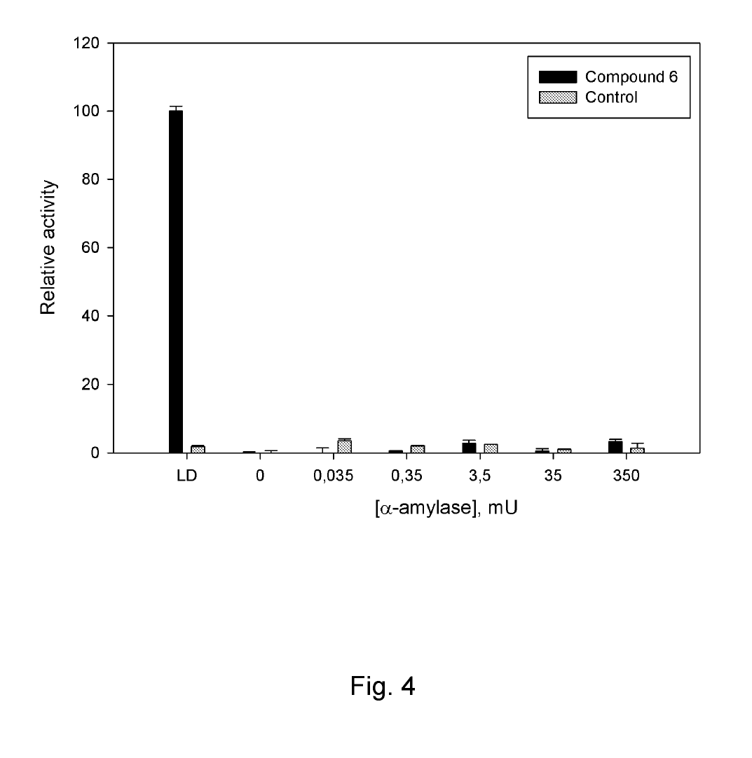

No o-NP was detected at any concentration of α-amylase suggesting that compound 6 is not a substrate for α-amylase (FIG. 4).

### Example 3

### Synthesis of TAMRA Labelled Heptasaccharide 9

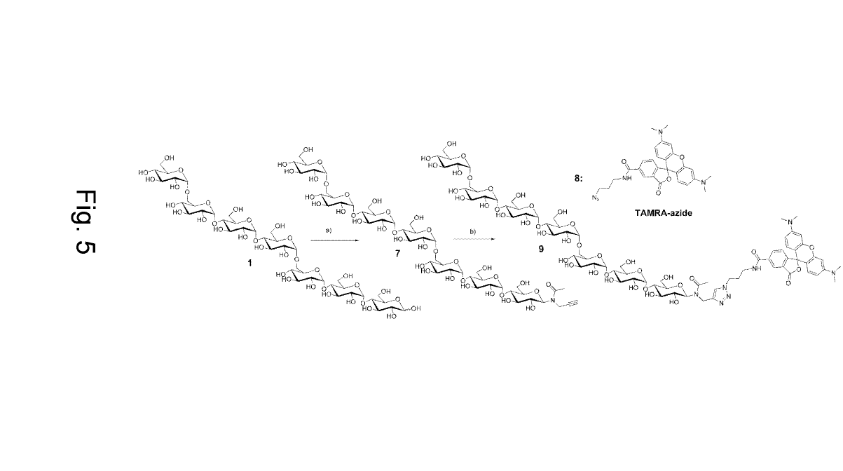

All compound numbers used in this example refers to the numbers indicated in FIG. 5. The heptasaccharide Glc-maltotriosyl-maltotriose compound 1 (Megazyme, Ireland) (FIG. 5) was protected. The reducing end of the oligosaccharide (compound 1) was first reacted with propargylamine followed by N-acetylation of the amine which resulted in the stable compound 7 (FIG. 5). The propargylic group allows the reducing end of the oligosaccharide to be functionalized with a variety of commercially available azide fluorophores via azide alkyne Huisgen cycloaddition. In order to initially determine the specificity of various hydrolytic enzymes towards the substrate, the tetramethyl rhodamine (TAMRA) labeled compound 9 was synthesized from compound 7 and the commercially available TAMRA-azide (compound 8) (Lumiprobe) catalyzed by Cu(I) (FIG. 5).

### A: Synthesis of Compound 7

6-α-D-Glucosyl-maltotriosyl-maltotriose (compound 1) (38.2 mg, 33.1 μmol) was dissolved in propargylamine (300 μL) and stirred at room temperature for 3 days. DCM/MeOH (3:1, 4.0 mL) was added and a white precipitate formed. The residue was centrifuged and the liquid removed. The remaining solid was washed with DCM/MeOH (3:1) (3×1.0 mL), centrifuged and decanted between washes. MeOH (4.0 mL) and Ac₂O (200 μL) was added to the solid after which the compound slowly dissolved. The mixture was left stirring at room temperature overnight. The mixture was evaporated and purified by reverse phase chromatography with increasing concentration of MeCN (100% H₂O to 30% MeCN in H₂O). Lyophilisation of the desired fractions gave compound 7 in a quantitative yield as a white solid.

¹H-NMR (400 MHz, D₂O): δ 5.62 and 5.16 (2×d, 1H, rotamers, J₁₋₂=9.4 Hz and J₁₋₂=8.8 Hz, H-1GlcI), 5.52-5.42 (m, 4H, H-1 GlcII, III, V, VI), 5.03 (br s, 2H, H-1 Glc IV, VII) 4.40-3.45 (m, 44H, H-2, 3, 4, 5, 6a, 6bGlcI-GlcVII, and NCH₂), 2.84 and 2.67 (2×s, 1H, rotamers, C≡CH), 2.40 and 2.32 (2×s, 3H, rotamers, NCOCH₃).

HRMS-ESI-ToF (m/z) Calcd. for C₄₇H₇₇NO₃₆N [M+Na]: m/z 1254.4123. Found: m/z 1254.4114 (NaI clusters used as internal lock mass).

### B: Synthesis of Compound 9

Propargylamine functionalised heptasaccharide (compound 7) (0.33 mg, 0.27 μmol) was dissolved in H₂O (30 μL). TAMRA-azide (compound 8) (10 mM in DMSO, 30 μL, 0.30 μmol) was added followed by isopropanol (30 μL) and CuSO₄ (50 mM in H₂O, 5 μL, 0.25 μmol), and Sodium Ascorbate (50 mM in H₂O, 14 μL, 0.7 μmol). The mixture was left at 50° C. for 15 min. Purified by preparative TLC, eluted with MeCN/H₂O (7.5:2.5). The lower fluorescent band was scraped off and extracted with MeCN/H₂O (1:1) then H₂O/Ethanol (1:1). Evaporated and purified by reverse phase chromatography eluting with increasing concentration of MeCN (100% H₂O to 30% MeCN). Fractions containing the desired compound were lyophilized to give compound 9 as a red powder (0.37 mg, 79%, >90% pure judged from UPLC analysis).

HRMS-ESI-ToF (m/z) Calcd. for C₇₅H₁₀₆N₇O₄₀ [M+1]: m/z 1744.6476. Found: m/z 1744.6460. (Renin substrate tetradecapeptide used as internal lock mass).

### Example 4

### Enzymatic Hydrolysis of TAMRA Labelled Heptasaccharide 9

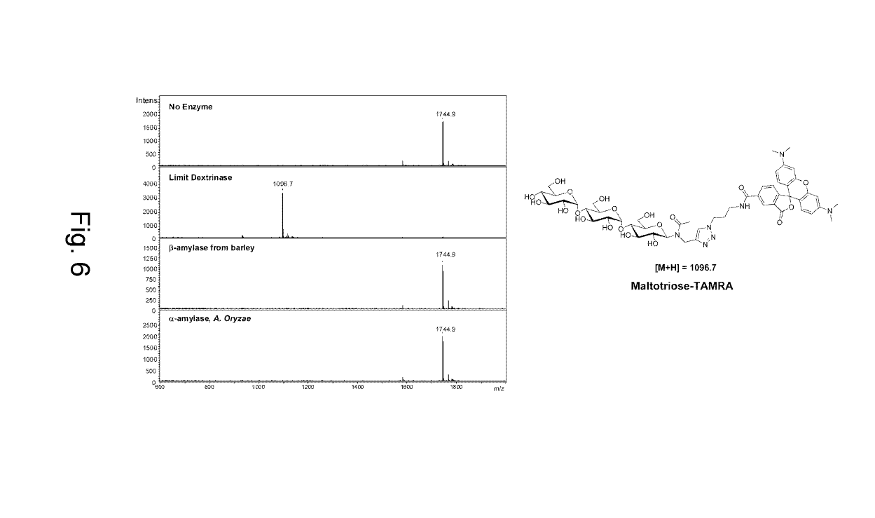

The fluorescence labeling of the oligosaccharide substrate facilitated easier detection by mass spectroscopy as well as the possibility to purify and detect the products. The TAMRA labeled heptasaccharide (compound 9, prepared as described in example 3 above) was treated with various glycoside hydrolases and the products after hydrolysis were analyzed by MALDI-ToF. He results are shown in FIG. 6.

A: Compound 9 (prepared as described in Example 3)(0.15 mg, 86 nmol) was dissolved in MilliQ water (12 μL), 1 μL (7.2 nmol) of this was used in each of the 5 entries.

B: 20 mM MES buffer pH 6.5.

The reactions were performed at 37° C. and quenched with 1 μL 1M Na₂CO₃ after 10 min.

1. A (1 μL), B (7 μL), no enzyme added

2. A (1 μL), B (7 μL), recombinant LD, produced as described in example 8 (0.3 μL of 25 mg/ml)

3. A (1 μL), B (7 μL), α-amylase From B. licheniformis (Sigma A3404, 0.3 μL of 25 mg/ml)

4. A (1 μL), B (7 μL), α-amylase from A. oryzae (Sigma 10065) (0.3 μL, 25 mg/mL)

5. A (1 μL), B (7 μL), β-amylase from barley (Sigma A7130, 0.3 μL of 25 mg/ml)

Recombinant LD was prepared as described in Example 2.

Each sample was diluted with 200 μL MilliQ water and purified by reverse phase chromatography with increasing concentration of MeCN (100% H₂O to 30% MeCN in H₂O).

The fractions containing fluorescent compounds were analysed with MALDI-ToF (HCCA as matrix).

The results are shown in FIG. 6. Only when reacting compound 9 with recombinant LD (prepared as described in Example 5 herein below) formation of TAMRA-maltotriose (m/z 1096.7) was seen, indicating that compound 9 is cleaved exclusively at the α-(1→6)-linkage between the maltotriose units by LD, but not by the other enzymes tested. Thus the compound 9 was not cleaved by β-amylase from barley and α-amylase from A. Oryzae as only starting material (m/z 1744.9) was recovered from the enzymatic hydrolysis mixtures.

### Example 5

### Synthesis of Bifunctionally Labelled FRET Substrate, Compound 14

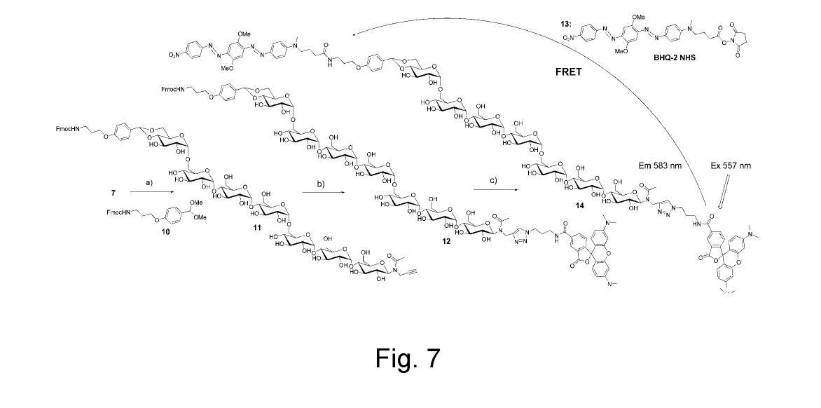

The compound number of this example refers to the compounds shown in the figures, mainly in FIG. 7. After having confirmed that the protected heptasaccharide (compound 9) was indeed a substrate for LD (see Example 4 above), the heptasaccharide was further protected in order to obtain a bifunctionally labeled FRET substrate (FIG. 7).

The non-reducing end of 7 was selectively functionalized with the dimethoxy acetal 10 by reacting 7 and 10 in the presence of camphor sulfonic acid (CSA) in DMF. The acetal protects the non-reducing end of compound 12 from hydrolysis by α-glucosidases present in malt as well as, after deprotection, provides an amine which can be derivatized with a variety of commercially available fluorophores (as their NHS esters). In this example the bifunctionally protected oligosaccharide (compound 9) was synthesized. Tetramethylrhodamine (TAMRA) was chosen as the donor and Black Hole Quencher-2 (BHQ-2) as the acceptor as the absorption spectrum of the acceptor (BHQ-2) overlaps with the fluorescence emission spectrum of the donor (TAMRA). The propargyl group of compound 11 was reacted with TAMRA-azide (compound 8) (Lumiprobe), catalyzed by Cu(I) as in example 3, giving the intermediate compound 12. The Fmoc-group of compound 12 was finally deprotected with DBU and the free amine reacted with the BHQ-2 NHS ester (compound 13) to give the bifunctionally labeled heptasaccharide (compound 14).

### A: Synthesis of Compound 11

Compound 7 (6.2 mg, 5 μmol) was dissolved in dry DMF (1.4 mL), dimethylacetal (compound 10) (see synthesis in example 7) (13 mg, 29 μmol) was added followed by freshly dried 4 Å MS and a catalytic amount of camphor sulfonic acid. The mixture was heated to 60° C. for 3½ hours. TLC (MeCN/H₂O, 7:3) showed satisfactory conversion. Triethylamine (5 μL) was added and the reaction purified by preparative TLC eluting with MeCN/H₂O (7.5:2.5). The desired band was scraped off and extracted with MeCN/H₂O (7:3). The eluent was evaporated and purified by reverse phase chromatography eluting with increasing concentration of MeCN (100% H₂O to 50% MeCN in H₂O). Fractions containing the desired compound were lyophilized to give compound 11 as a white powder (1.36 mg, 17%, 80% pure judged from UPLC analysis).

HRMS-ESI-ToF (m/z) Calcd. for C₇₂H₉₈N₂O₃₉Na [M+Na]: m/z 1637.5645. Found: m/z 1637.5691 (Renin substrate tetradecapeptide used as internal lock mass)

### B: Synthesis of Compound 12

Compound 12 was synthesised according to the procedure described in example 3, synthesis B.

### C: Synthesis of Compound 14

### Solution A:

Compound 12 (0.1 mg, 47 nmol) was dissolved in DMF (100 μL) and DBU (2 μL) was added. The mixture was left at room temperature for 1 hour. MALDI-ToF showed completion of reaction.

### Solution B:

BHQ-2 NHS ester (Biosearch Technologies) (compound 13) (0.97 mg, 1.6 μmol) was dissolved in dry DMSO (100 μL) giving a solution of 16 mM.

### Solution C:

Phosphate buffer, Na₂HPO₄ (3 g) and NaH₂PO₄ (0.44 g) in MilliQ water (200 mL), pH 7.6.

Solution C (0.5 mL) was added to solution A followed by solution B (7 μL, 0.11 μmol). The mixture was left at room temperature for 30 min then extra solution A (10 μL, 0.16 μmol) was added (17 μL solution A in all, 0.27 μmol). The mixture was left at room temperature for 2 hours. MilliQ water was added until 2 mL, after which the mixture was purified by reverse phase chromatography eluting with increasing concentration of MeCN (100% H₂O to 50% MeCN in H₂O). The fractions containing the desired compound were lyophilized to give compound 14 as a purple solid (0.05 mg, 44%). MALDI-ToF (m/z) Calcd. for C₁₁₀H₁₄₁N14O₄₆ [M+1]: m/z 2393.91. Found: m/z 2394.44.

### Example 6

### Enzymatic Hydrolysis of FRET Substrate (Compound 14)

A: Compound 14 (prepared as described herein above in Example 5) (0.05 mg, 21 nmol) was dissolved in Na-maleate buffer (0.2M pH 5.5, 0.02% NaN₃ 200 μL).
1: A (100 μL), recombinant limit dextrinase from P. Pastoris (prepared as described in Example 5) (2 μL, 3.9 mg/mL based on ε_{280 nm}=1.52*10⁵ M⁻¹ cm⁻¹))
2: A (100 μL), no enzyme added
3: Na-maleate buffer (0.2M pH 5.5, 0.02% NaN₃ 100 μL), recombinant limit dextrinase from P. Pastoris (2 μL, 3.9 mg/mL)

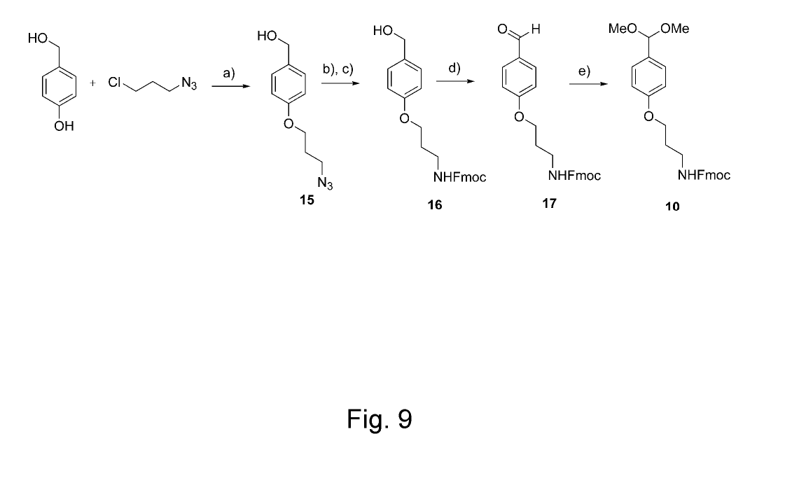

The enzyme was added and immediately after the fluorescence intensity was measured on a fluorescence plate reader, SpectraMax Gemini EM, Molecular Devices every 5 minutes for 2 hours. Excitation 557 nm; emission 583 nm. The results are shown in FIG. 9.

Compound 14 prepared as described in Example 5 herein above was reacted with recombinant LD in maleate buffer at pH 5.5, 40° C. (FIG. 9). Excitation was performed at 557 nm and the change in fluorescence at the maximum emission wavelength of TAMRA (583 nm) was monitored over time. An increase in fluorescence intensity was indeed observed showing, in real-time, the hydrolysis of compound 14 by LD (FIG. 9). No increase in emission was observed in the absence of LD.

### Example 7

### Synthesis of Dimethoxy Acetal, Compound 10

The dimethoxyacetyl compound 10 was synthesized in order to protect the non-reducing sugar as well as provide a protected amine to the oligosaccharide. All compound numbers refers to the numbers indicated in FIG. 9.

### A: Synthesis of Compound 15

### 4-(3-azido-propoxy)-benzylalcohol (15)

1-azido-3-chloropropane (0.67 g, 5.6 mmol) (synthesized according to Verdoes, M. Bogdan, I. Florea, U. H, Hillaert, U. Willems, L. I. van der Linden, W. A. Sae-Heng, M. Filippov, D. V. Kisselev, A. F. van der Marel, G. A. Overkleeft, H. S. Chembiochem. 2008, 9, 1735) was dissolved in dry DMF (5 mL). 4-hydroxy-benzylalcohol (0.35 g, 2.8 mmol) was added followed by powdered K₂CO₃ (0.77 g, 5.6 mmol). The mixture was heated to 80° C. over night under argon. The mixture was cooled and diethyl ether (30 mL) added. The organic phase was washed with sat. NaHCO₃ (20 mL), H₂O (20 mL) and brine (20 mL), dried (Na₂SO₄), filtered and evaporated. The residue was purified by flash chromatography (EtOAc/Hexane, 1:1) to give 15 as a colourless oil (0.46 g, 78%).

¹H-NMR (400 MHz, CDCl₃): δ 7.31-7.27 (m, 2H), 6.91-6.87 (m, 2H), 4.62 (s, 2H), 4.05 (t, 2H, J=5.9 Hz), 3.52 (t, 2H, J=6.6 Hz), 2.05 (m, 2H).

¹³C-NMR (100 MHz, CDCl₃): δ 158.4, 133.5, 128.8, 114.7, 65.2, 64.7, 48.4, 28.9.

### B: Synthesis of Compound 16

### 4-(3-(((9H-fluoren-9-ylmethoxy)carbonyl)amino)propoxy)-benzylalcohol (16)

Compound 15 (0.42 g, 2.03 mmol) was dissolved in MeOH (7 mL) and a cat., amount of Pd/C (10% Pd) was added. The mixture was stirred under hydrogen for 1 hour, and then filtered through Celite. The mixture was evaporated in vacuo to give the amine in a quantitative yield as a white solid. This was used in the next step without further purification. The amine (0.36 g, 1.99 mmol) was dissolved in dioxane (10 mL) and Na₂CO₃ (10%, 10 mL) was added. The mixture was cooled to 0° C. on an ice bath. FmocCl (565 mg, 2.19 mmol) was added at this temperature and stirred for 2 hours. The mixture was extracted with diethyl ether and the pooled organic phases washed with H₂O (2×20 mL), brine (20 mL), dried (Na₂SO₄), filtered and evaporated. Purified by flash chromatography (EtOAc/Hexane, 1:1, then 6:4) to give compound 16 as a white solid (591 mg, 73%).

¹H-NMR (400 MHz, CDCl₃): δ 7.76 (d, J=7.5 Hz, 2H), 7.59 (d, J=7.5 Hz, 2H), 7.40 (t, J=7.5, 2H), 7.32-7.28 (m, 4H), 6.88 (d, J=8.5, 2H), 5.01 (br s, 1H), 4.62 (s, 2H), 4.42 (d, J=6.5, 2H), 4.21 (t, J=6.5, 1 H), 4.04 (br m, 2H), 3.41 (br m, 2H), 2.00 (br m, 2H).

¹³C-NMR (100 MHz, CDCl₃): δ 158.5, 156.6, 144.1, 141.5, 133.6, 128.8, 127.8, 127.16, 125.2, 120.1, 114.7, 66.7, 66.0, 65.1, 47.5, 38.8, 29.5.

### 4-(3-(((9H-fluoren-9-ylmethoxy)carbonyl)amino)propoxy)-benzaldehyde (17)

Compound 16 (111 mg, 0.275 mmol) was dissolved in dry THF (5 mL). Activated MnO₂ (0.40 g, 4.60 mmol) was added and the mixture stirred at room temperature for 30 min. Filtered through Celite and evaporated to give the crude product as a yellow oil. This was purified by flash chromatography (EtOAc/Hexane, 6:4) to give compound 17 as a white solid (78 mg, 73%)

¹H-NMR (400 MHz, CDCl₃): δ 9.89 (s, 1H), 7.83 (m, 2H), 7.76 (m, 2H), 7.58 (m, 2H), 7.40 (m, 2H), 7.30 (m, 2H), 6.98 (m, 2H), 4.94 (m, 1H), 4.43 (d, 2H, J=6.4 Hz), 4.21 (t, 1H, J=6.4 Hz), 4.11 (m, 2H), 3.41 (m, 2H), 2.05 (m, 2H).

¹³C-NMR (100 MHz, CDCl₃): δ 190.8, 163.9, 156.6, 144.1, 141.5, 132.1, 130.3, 127.85, 127.2, 125.1, 120.1, 114.9, 66.7, 66.2, 47.5, 38.5, 29.5.

### 4-(3-(((9H-fluoren-9-ylmethoxy)carbonyl)amino)propoxy)-benzaldehyde-dimethylacetal (10)

Compound 17 (86 mg, 0.21 mmol) was dissolved in dichloromethane (3 mL) and methanol (3 mL). Trimethylorthoformate (250 μL, 2.3 mmol) was added together with a catalytic amount of p-toluenesulfonic acid. The mixture was stirred at room temperature over night under argon. Triethylamine (3 μL) was added and the mixture evaporated in vacuo. The mixture was dissolved in EtOAc (10 mL), washed with H₂O (2 times 10 mL), brine (10 mL), dried (Na₂SO₄), filtered and evaporated in vacuo to give compound 10 as a white solid (92 mg, 95%). This was used without further purification in the reaction with compound 7.

¹H-NMR (400 MHz, DMSO-d₆): δ 7.87 (d, 2H, J=7.5), 7.67 (d, 2H, J=7.3 Hz) 7.40 (t, 2H, J=7.3 Hz), 7.33-7.27 (m, 5H), 6.90 (d, 2H, J=8.7 Hz), 5.30 (s, 1H), 4.31 (d, 2H, J=6.8 Hz), 4.20 (t, 1H, J=6.6), 3.98 (br m, 1H), 3.21 (s, 6H), 3.15 (br m, 2H), 1.85 (br m, 2H).

¹³C-NMR (100 MHz, DMSO-d₆): δ 158.5, 156.1, 143.9, 140.7, 130.3, 127.7, 127.5, 127.0, 125.0, 120.0, 113.9, 102.5, 65.2, 65.0, 52.3, 46.7, 37.2, 29.0.

### Example 8

### Recombinant Expression of Limit Dextrinase

The barley limit dextrinase gene (HvLD) codon optimized for Pichia pastoris from GenScript was cloned into the pPinkα-HC vector. The pPinkα-HC-HvLD vector was linearized with Afl II and transformed into PichiaPink strain 1 from Invitrogen. Screening for colonies were done as described in the Invitrogen user manual[1]. The expression level in Pichia Pink was at the same level as described by Vester-Christensen et al. [2].
[1] PichiaPink™ Expression system, Version A, 15 Jan. 2009, A10984. Invitrogen.
[2] Vester-Christensen, M. B., Hachem, M. A., Naested, H., and Svensson, B., (2010) Protein Expr. Purif. 69, 112-119

### Example 9

### Synthesis of Chromogenic Substrate 19

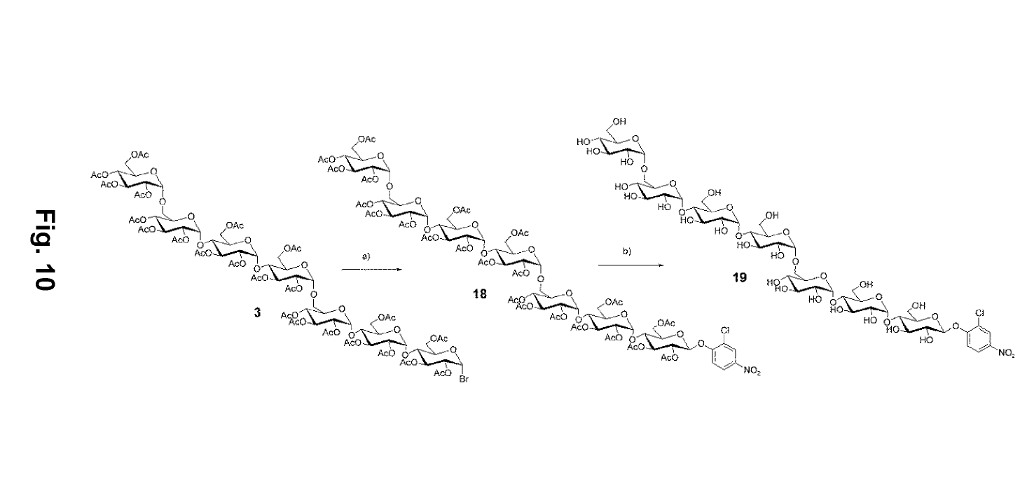

The reaction scheme for the synthesis of 19 is illustrated in FIG. 10 and the compound numbers refer to the numbers indicated in FIGS. 1 and 10.

The bromosugar 3 (53 mg, 25 μmol) was dissolved in dichloromethane (2 ml) and aqueous NaOH (0.75 M, 1 mL). 2-chloro-4-nitrophenol (14 mg, 80 μmol) and tetrabutyl ammonium bromide (TBABr) (8 mg, 24.8 μmol) was added and the mixture stirred vigorously at room temperature over night. DCM (5 mL) was added and the organic phase washed with saturated NaHCO₃, brine, dried (Na₂SO₄), filtered and evaporated. The residue was purified by flash chromatography (EtOAc/Hexane, 2:1 then 4:1). Fractions containing the desired compound were evaporated to give the desired compound 18 as a colorless oil (11 mg, 20%).

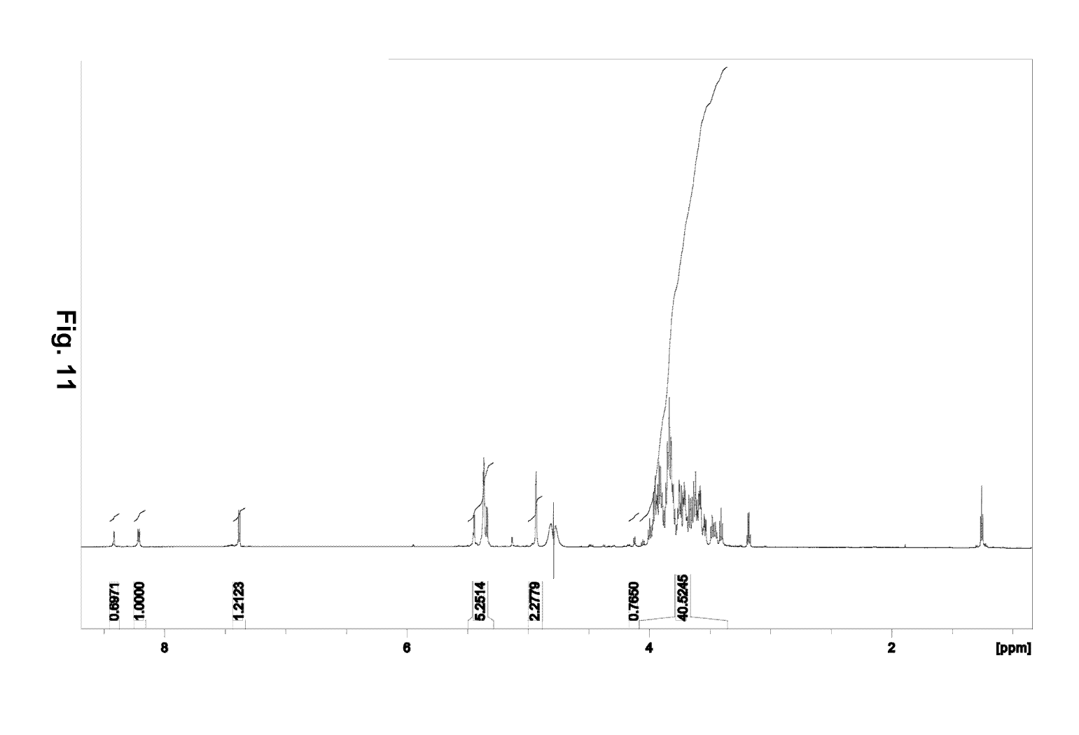

The residue from above was dissolved in MeOH (2 mL), Et₃N (2 mL) and H₂O (1 mL). The mixture was left stirring 2 days. The mixture was evaporated and passed through mixed bed resin (MB-1) to remove salts. The fractions containing compound were evaporated to give the desired compound as a white powder (2.4 mg, 37%). (The overall yield was 7.4%). ¹H NMR spectra of compound 19 is shown in FIG. 11.

### Example 10

### Investigation of the Specificity of Starch Hydrolytic Enzymes Present in Barley Malt Extract Towards Compound 19

It was envisaged that compound 19 could be a selective substrate for limit dextrinase in barley malt extract even in the presence of other starch hydrolytic enzymes. This is only the case if 19 is not cleaved by exo-glucosidases present in the malt extract such as β-amylase and α-glucosidases, i.e. if the glucoside at the non-reducing end serves as a blocking group when linked via a (1→6)-glucosidic linkage. This was investigated by enzymatic hydrolysis of the substrate in the presence and absence of barley limit dextrinase inhibitor (LDI, produced according to Jensen et al., 2011). Barley limit dextrinase is selectively inhibited by the endogenous barley limit dextrinase inhibitor (LDI) which is also present in the malt extract causing a decrease in the measured LD activity. This inhibitor can be deactivated by addition of 25 mM DTT in the extract due to reduction of disulfide bridges in the inhibitor. Extracts containing DTT will therefore show an increased LD activity compared to extracts containing no DTT.

Buffer: 100 mM NaOAc buffer pH 5.33, 5 mM CaCl₂

Stock sample: 1 mg compound 19 prepared as described in Example 9 was dissolved in 191 μL buffer (4 mM).

Extract A: 0.25 g grinded malt was extracted with 2 mL buffer for 30 min according to standard procedure.

Extract B: 0.25 g grinded malt was extracted with 2 mL buffer containing 7.5 mg DTT (app. 25 mM) for 30 min according to standard procedure.

Four different samples were prepared for enzymatic hydrolysis and UPLC analysis.
1) Substrate: 12.5 μL stock sample was diluted in 47.5 μL buffer. The mixture was heated to 60° C. for 1 h.
2) Extract+LDI: 20 μL A was placed in an eppendorf tube, 16.5 μL buffer and 1 μL LDI (recombinant barley limit dextrinase inhibitor, 4.7 mg/mL) was added and left for a few minutes at room temperature. 12.5 μL stock sample was added and the mixture heated to 60° C. for 1 h.
3) Extract: 20 μL A was placed in an eppendorf tube, 17.5 μL buffer and left for a few minutes at room temperature. 12.5 μL stock sample was added and the mixture heated to 60° C. for 1 h.
4) Extract (25 mM DTT): 20 μL B was placed in an eppendorf tube, 17.5 μL buffer and left for a few minutes at room temperature. 12.5 μL stock sample was added and the mixture heated to 60° C. for 1 h.

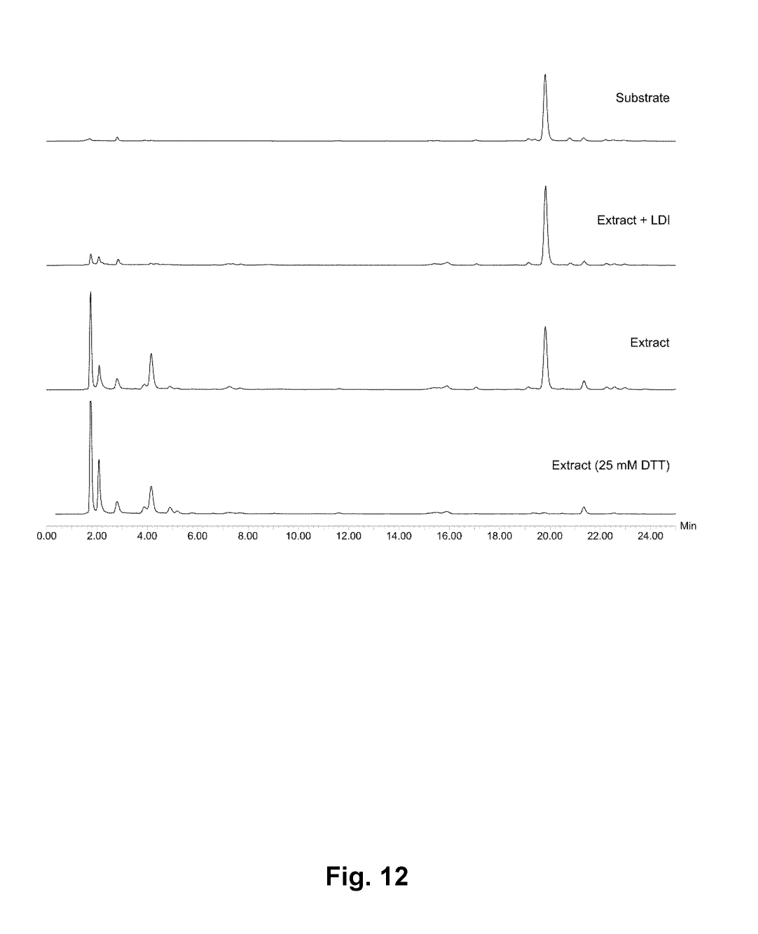

Aliquots (10 μL) from the above samples were taken after 1 hour, diluted in 190 μL running buffer. The UPLC chromatogram (fluorescence detection) of the four samples can be seen in FIG. 12.

Separation was performed on an Acquity UPLC system using an Acquity UPLC BEH glycan 1.7 μm, 2.1×150 mm column with a VanGuard BEH glycan 1.7 μm, 2.1×5 mm pre-column at room temperature. 5 μL of the samples were injected. Fluorescence detection: Excitation Wavelength: 320 nm, Emission Wavelength: 420 nm.

Solvent A: 10 mM Ammonium formate pH 4.5

Solvent B: Acetonitrile

TABLE 1

| Time (min) | Flow Rate (mL/min) | % A | % B | Curve |
|---|---|---|---|---|
| Initial | 0.200 | 22.0 | 78.0 | |
| 5.00 | 0.200 | 22.0 | 78.0 | 6 |
| 38.00 | 0.200 | 45.0 | 55.0 | 6 |
| 39.00 | 0.200 | 70.0 | 30.0 | 6 |
| 44.00 | 0.200 | 22.0 | 78.0 | 6 |
| 45.00 | 0.200 | 22.0 | 78.0 | 6 |

The results are shown in FIG. 12. When extract containing no DTT was added, compound 19 was hydrolyzed partly after 1 h giving mainly labeled maltotriose, maltose, glucose and 2-chloro-4-nitrophenol. When extract containing 25 mM DTT was added, no substrate was left after 1 hour corresponding to the increased activity of limit dextrinase in an extract containing DTT. When LDI was added to the extract, almost all activity of limit dextrinase was inhibited, and only negligible amounts of the compound 19 was hydrolyzed meaning that 19 is indeed primarily hydrolyzed by the limit dextrinase present in barley malt extract and not by the starch exo-glucosidases present. Accordingly, the glucoside at the non-reducing end serves as a blocking group when linked via a (1→6)-glucosidic linkage.

### Example 11

### Assay for Limit Dextrinase Using Compound 19 as Chromogenic Substrate

In order to release the chromogenic group from the reducing end of compound 19, additional enzymes may be used to fully hydrolyse the oligosaccharide formed after the initial hydrolysis with limit dextrinase. In this example an activity of externally added α-glucosidase of 30 U/mL and an activity of externally added β-glucosidase of 15 U/mL was chosen after optimization. In this example the linearity of the assay using barley malt extract is shown.

Buffer: 100 mM NaOAc buffer pH 5.33, 5 mM CaCl₂.

α-glucosidase: (Megazyme E-TSAGL, 750 U/mL), 0.75 U/μL, 2 μL to each well, (30 U/mL in working solution).

β-glucosidase: (Megazyme E-BGOSAG, 380 U/ml), 0.38 U/μL, 2 μL to each well, (15.2 U/mL in working solution).

Substrate: 1.71 mg of compound 19 prepared as described in Example 9 was dissolved in 0.262 ml buffer (stock 5 mM). 5 μL substrate was added to each well (working solution 0.5 mM).

Malt extract: Grinded malt was extracted with buffer containing 25 mM DTT for 30 min according to standard procedure. 0-25 μL extract was added to each well, corresponding to 0-50% extract in assay.

50 μL total in each well (NUNC 96 well plate, half area). Two replicates were made. Equilibrated at 45° C. for 3 min in the plate reader before addition of substrate. Absorption (405 nm) was measured every 20 seconds in a SpectraMax 340PC384 Absorbance Microplate Reader from Molecular Devices.

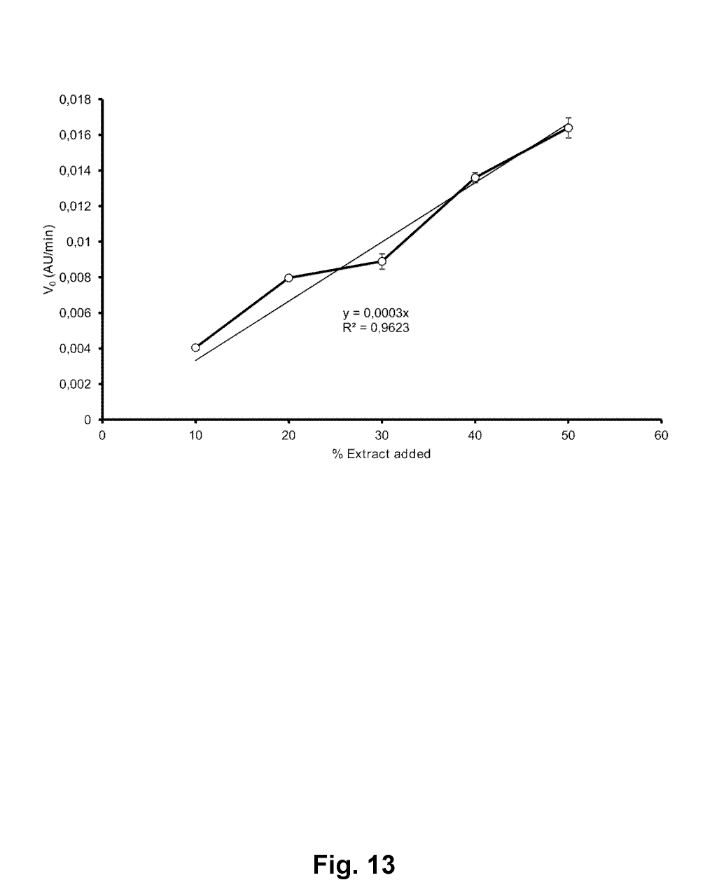

The results are shown in FIG. 13. As seen from this figure the initial velocity of the hydrolysis of compound 19 with barley malt extract is linearly dependend on the amount of extract added.

### Example 12

### Enzyme Kinetics for Limit Dextrinase from Barley Malt Extract

The chromogenic substrate 19 can be used to perform an enzyme kinetic characterization of barley limit dextrinase from barley malt extract as shown below.

Buffer: 100 mM NaOAc buffer pH 5.33, 5 mM CaCl₂.

α-glucosidase: (Megazyme E-TSAGL, 750 U/mL), 0.75 U/μL, 2 μL to each well, (30 U/mL in working solution).

β-glucosidase: (Megazyme E-BGOSAG, 380 U/ml), 0.38 U/μL, 2 μL to each well, (15.2 U/mL in working solution).

Substrate: 1.43 mg of compound 19 prepared as described in Example 9 was dissolved in 0.219 ml buffer (stock 5 mM), 0-10 μL was added to each well corresponding to 0-1 mM.

Malt extract: Grinded malt was extracted with buffer containing 25 mM DTT for 30 min according to standard procedure. 20 μL extract was added to each well (corresponding to 40% extract in assay).

50 μL total in each well (NUNC 96 well plate, half area). Two replicates were made. Equilibrated at 45° C. for 3 min in the plate reader before addition of substrate. Absorption (405 nm) was measured every 20 seconds in a SpectraMax 340PC384 Absorbance Microplate Reader from Molecular Devices.

2-chloro-4-nitrophenol was used as standard to determine the relationship between absorption and concentration of the chromophore. A non-linear regression analysis (GraphPad Prism version 4.02 for Windows, GraphPad Software, San Diego Calif., USA) was used to determine Michaelis-Menten parameters (Kₘ and Vₘₐₓ values). The result can be seen in FIG. 14.

### REFERENCES

Vester-Christensen M B, Hachem M A, Naested H, Svensson B. (2010) Protein Expr Purif 69(1):112-9.
Jensen J M, Vester-Christensen M B, Møller M S, Bønsager B C, Christensen H E, Hachem M A, Svensson B (2011) Protein Expr Purif 79(2):217-22

### ABBREVIATIONS

AcOH: Acetic acid
BHQ: Black hole quencher
CSA: Camphor sulfonic acid
Cy 3: Cy 3™, cyanine dye
Cy 5: Cy-5™, cyanine dye
DABCYL: 4,4-Dimethylamino-azobenzene-4′-carboxylic acid

### DCM: Dichloromethane

DMAP: Dimethyl aminopyridine
DMF: dimethylformamide

### DTT: Dithiothreitol

EDANS: 5-((2-aminoethyl)amino)naphthalene-1-sulfonic acid
ESI-MS: Electrospray ionisation mass spectrometer

### EtOAc: Ethylacetate

FRET: Fluorescence resonance energy transfer

### Glc: Glucose

HCCA: α-cyano-4-hydroxycinnamic acid

### LD: Limit Dextrinase

LDI: Limit dextrinase inhibitor
MALDI-ToF-MS: Matrix-assisted laser desorption/ionization mass spectrometer
Me: methyl

### MeCN: Acetonitril

### MeOH: Methanol

NMR: Nuclear magnetic resonance
ON: Over night
Q-ToF: Quadrupole time-of-flight mass spectrometer
Rt: Room temperature

### TAMRA: Tetramethylrhodamine

TBABr: Tetrabutylammonium bromide
TLC: Thin layer chromatography

1. A method of detecting α-(1→6)-glucosidase activity in a sample, the method comprising the steps of
a. Providing a sample;
b. Providing an oligosaccharide substrate of the formula
X-(glucoside)ₙ-*(glucoside)ₘ-Z—Y
wherein X is a blocking group capable of inhibiting cleavage by an exo-glucosidase; and
Y is a detectable label; and
Z is either S or O or N; and
-* is an α-(1→6)-glucosidic linkage; and
n and m individually are integers in the range of 1 to 6;
c. Optionally, providing at least one exo-glucosidase;
d. Incubating the sample with the oligosaccharide substrate and optionally with the exo-glucosidase;
e. Determining the presence of free detectable label,
wherein the presence of free detectable label is indicative of α-(1→6)-glucosidase activity in said sample.

2. The method according to claim 1, wherein the method is a method of detecting limit dextrinase activity in a sample, the method comprising the steps of
a. Providing a sample;
b. Providing an oligosaccharide substrate of the formula
X-(glucoside)ₙ-*(glucoside)ₘ-Z—Y
wherein X is a blocking group capable of inhibiting cleavage by an exo-glucosidase; and
Y is a detectable label; and
Z is either S or O or N; and
-* is an α-(1→6)-glucosidic linkage; and
n and m individually are integers in the range of 1 to 6;
c. Optionally providing at least one exo-glucosidase;
d. Incubating the sample with the oligosaccharide substrate and optionally with the exo-glucosidase;
e. Determining the presence of free detectable label,
wherein the presence of free detectable label is indicative of limit dextrinase activity in said sample.

3. The method according to claim 1, wherein step c) comprises providing at least one exo-glucosidase and step d) comprises incubating the sample with the oligosaccharide substrate and with the exo-glucosidase.

4. An oligosaccharide substrate of the formula I
X-(glucoside)ₙ-*(glucoside)ₘ-Z—Y
wherein X is a blocking group capable of inhibiting cleavage by an exo-glucosidase; and
Y is a detectable label; and
Z is either S or O or N; and
-* is an α-(1→6)-D-glucosidic linkage; and
n and m individually are integers in the range of 1 to 6.

5. The oligosaccharide substrate according to claim 4, wherein the substrate is a compound of formula II, formula III, formula IV or formula V; wherein formula II is 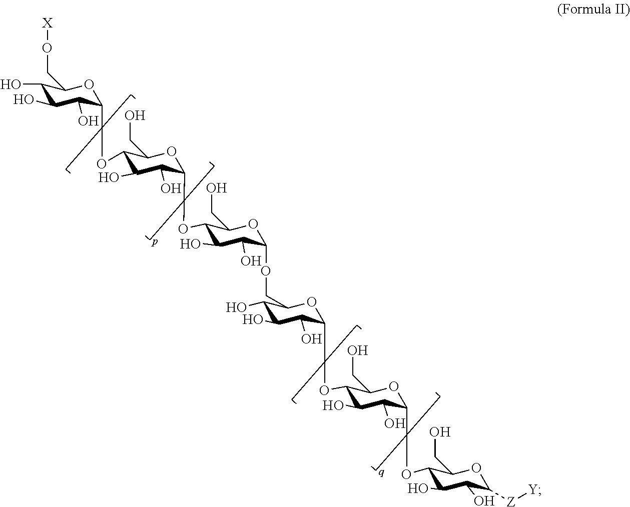
wherein formula III is: 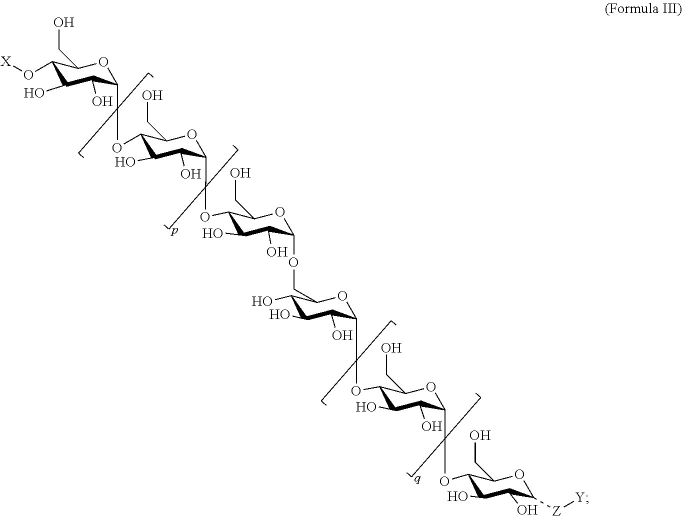
wherein formula IV is: 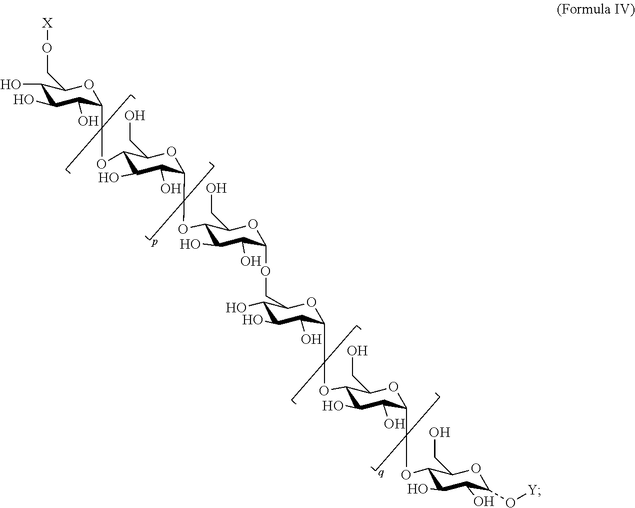
wherein formula V is: 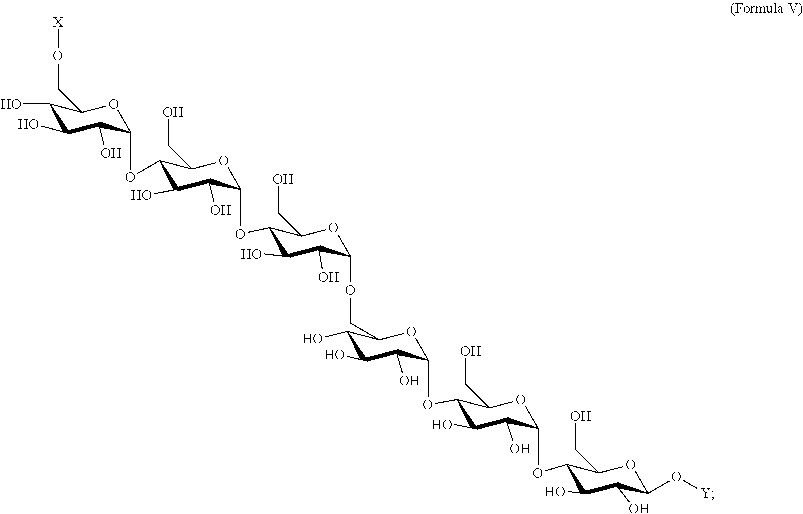
wherein X and Y is as defined in claim 4; and
Z is either N or O or S; and
p and q individually are integers in the range of 0 to 4 and
wherein the dotted line indicates a bond, which may be either in the α or the β configuration.

6-8. (canceled)

9. The oligosaccharide substrate according to claim 4, wherein X has the structure 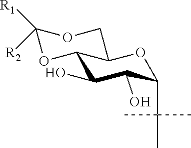
wherein R₁ and R₂ individually are selected from the group consisting of —H, alkyl, aryl, heteroaryl and —COOH, wherein any of the aforementioned may optionally be substituted with one or more substituents; and
the dotted line indicates the point of attachment.

10. The oligosaccharide substrate according to claim 9, wherein R₁ and R₂ individually are selected from the group consisting of H, alkyl, aryl and heteroaryl, wherein any of the aforementioned may optionally be substituted with one or more substituents selected from the group consisting of alkyl, alkoxy and amino-alkyl.

11. The oligosaccharide substrate according to claim 9, wherein R₁ and R₂ individually are selected from the group consisting of H, alkyl, aryl and heteroaryl, wherein any of the aforementioned is substituted with a fluorophore or a quencher capable of quenching a fluorescent signal.

12. The oligosaccharide substrate according to claim 9, wherein at the most one of R₁ and R₂ is —H and at least one of R₁ and R₂ is —H.

13. The oligosaccharide substrate according to claim 4, wherein X has the structure 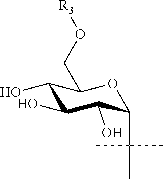
wherein R₃ is selected from the group consisting of alkyl, —(C═O)-alkyl, —(C═O)-aryl, —(C═O)-heteroaryl, aryl, heteroaryl, -alkyl-COOH, alkenyl, phosphate esters, sulfonate esters and glycoside, wherein any of the aforementioned may optionally be substituted with one or more substituents and with the proviso that said glycoside is not a glucoside; and
the dotted line indicates the point of attachment.

14. The oligosaccharide substrate according to claim 4, wherein X is R₃, wherein R₃ is selected from the group consisting of alkyl, —(C═O)-alkyl, —(C═O)-aryl, —(C═O)-heteroaryl, aryl, heteroaryl, -alkyl-COOH, alkenyl, phosphate esters, sulfonate esters and glycoside, wherein any of the aforementioned may optionally be substituted with one or more substituents and with the proviso that said glycoside is not a glucoside.

15. The oligosaccharide substrate according to anyone of claims 13 to 14, wherein R₃ is C₁₋₅ alkyl, such as C₁₋₃ alkyl.

16. The oligosaccharide substrate according to anyone of claims 13 to 14, wherein R₃ is 5 to 10 membered aryl or 5 to 10 membered heteroaryl or phenyl.

17. The oligosaccharide substrate according to anyone of claims 13 to 14, wherein R₃ is a fluorophore or a quencher capable of quenching a fluorescent signal.

18. The oligosaccharide substrate according to claim 5, wherein X is glucoside.

19. The oligosaccharide substrate according to claim 5, wherein X has the structure 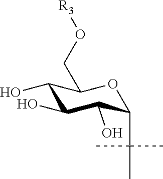
and wherein R₃ is —H.

20. The oligosaccharide substrate according to claim 4, wherein Y or Y in its free form Y—OH is selected from the group consisting of chromophores, fluorophores and haptens.

21. The oligosaccharide substrate according to claim 4, wherein Y is nitrophenyl optionally substituted with one or more groups selected from the group consisting of hydroxyl, nitro, —(CH₂)_{P}—(C═O)—R₄ and halogen, wherein p is an integer in the range of 0 to 4 and R₄ is selected from the group consisting of alkoxy, —NH-alkyl, —NH2 and —OH.

22. The oligosaccharide substrate according to claim 4, wherein Y is selected from the group consisting of umbelliferyl, 4-methyl umbelliferyl, 1-naphtyl, o-nitrophenyl, m-nitrophenyl, p-nitrophenyl, 2,4-nitrophenyl, 4-chloro-2-nitrophenyl, 4-hydroxy-3-nitrophenylacetic acid esters and 4-hydroxy-3-nitrophenylacetic acid amides.

23. The oligosaccharide substrate according to claim 4, wherein Y or Y—OH is a fluorophore.

24. The oligosaccharide substrate according to claim 4, wherein Y is a fluorophore and X comprises a quencher capable of quenching the fluorescence of said fluorophore.

25. The oligosaccharide substrate according to claim 4, wherein X comprises a fluorophore and Y comprises a quencher capable of quenching the fluorescence of said fluorophore.

26. The oligosaccharide substrate according to claim 4, wherein Y or Y—OH is a hapten.

27. (canceled)

28. The method according to claim 1, wherein Y or Y—OH is a hapten and step e. of the method comprises detecting the free hapten with an antibody specifically recognising the free hapten or with an antibody specifically recognising the free hapten and/or the hapten conjugated or attached to a protein or the hapten conjugated or attached to a surface.

29. The method according to claim 1, wherein Y is a chromophore in its free form Y—OH or Y—O⁻ and step e. of the method comprises detecting the absorption intensity of said chromophore.

30. The method according to claim 1, wherein Y is a fluorophore in its free form Y—OH or Y—O⁻ and step e. of the method comprises detecting the emission intensity of said fluorophore.

31. The method according to claim 1, wherein Y is a fluorophore and X comprises a quencher capable of quenching the fluorescence intensity of said fluorophore and step e. of the method comprises detecting fluorescence intensity of said fluorophore.

32. The method according to claim 1, wherein X comprises a fluorophore and Y comprises a quencher capable of quenching the fluorescence intensity of said fluorophore and step e. of the method comprises detecting fluorescence intensity of said fluorophore.

33. The method according to claim 1, wherein the sample comprises barley or an extract thereof.

34. The method according to claim 33, wherein the sample comprises barley, malt and/or wort.

35. The method according to claim 1, wherein the exoglucosidase is selected from the group consisting of glucan 1,4-α-glucosidase, α-glucosidase, β-glucosidase and β-amylase.

36. The method according to claim 1, wherein two different exo-glucosidases are used in steps c. and d.

37. The method according to claim 1, wherein step d) comprises incubating said sample with the oligosaccharide substrate in the presence of a reducing agent.
